SEQUENCE LISTING (16 sequences)

SEQ ID NO: 1 - protein, 9 aa, Unknown
  Description of Unknown Naturally-occurring meganuclease
LAGLIDADG

SEQ ID NO: 2 - protein, 7 aa, Unknown
  Description of Unknown Nuclear localization signal peptide
PKKKRKV

SEQ ID NO: 3 - protein, 7 aa, Unknown
  Description of Unknown Nuclear localization signal peptide
PKKKRRV

SEQ ID NO: 4 - protein, 16 aa, Unknown
  Description of Unknown Nuclear localization signal peptide
KRPAATKKAGQAKKKK

SEQ ID NO: 5 - protein, 6 aa, Artificial Sequence
  Description of Artificial Sequence Synthetic 6xHis tag
HHHHHH

SEQ ID NO: 6 - DNA, 20 bp, Artificial Sequence
  Description of Artificial Sequence Synthetic oligonucleotide
aaggaggttaggcatgtctc

SEQ ID NO: 7 - DNA, 21 bp, Artificial Sequence
  Description of Artificial Sequence Synthetic primer
gtgggcagtttgttcaatctg

SEQ ID NO: 8 - DNA, 17 bp, Artificial Sequence
  Description of Artificial Sequence Synthetic oligonucleotide
ttcgcggccgcacgcgt

SEQ ID NO: 9 - DNA, 4163 bp, Artificial Sequence
  Description of Artificial Sequence Synthetic polynucleotide
aggaggtcatgatccccttctggtcttccttcagtctgtaaacctcagaacttgtagctaatgctaaacaaaaaagccacatttatcaatgtgtacttaaaatccttaattcagacaacaggaatattttgagaatgagttccctattcctcacttggtcaaaatggaagcaaatgtaagagaagaatgacattaaggcacaatgcagaggcacttctgtttgtcttcttttatttgaaaagtatgcatatgtattctgtatttatcttttggccagtatgttgggcaaagaaacataagtgcttactttactgtctttattagtaggaatataaccttcatattcctgtggtgaccttatgttaaattaggaggagtaccagaggctagaaattatgagatgtcctacttgagcacaggtgcagctaggcagggctctctcaatattatttcacctagcacatctgggagttactccagatcttccccctcaatattcagcctgggtagggttgaaataaatttaacctgagttcactggatttttgcactttatcaaaatctgttccaatattctacactcaaattaaaatctattttttgattctctgtggctttaagttcattaaatgtaaaattggcagcttgctaaagaaggtcagactgattaactgtttaagacttgtacattttctgcttcagttttattaactggcagcatcctggatgttttgtattttgtgattttttttttttttttgatagagcaagcataagatttcacaagcagagacttaccaactctcttttcccctttggaagcttaaaaaatgatagaagctggtaaagtagatgctggagtattttagtacaaagttaaaaaaaaaagcaaacaggaaagaaagacatgtctaccttgttataccatccgctggtgattatgtgtgcagaaatagtctcataatgaagcattttggagctcattcagaaaattagtccactttgacaacattaggcgaagtatttcaagtctaaagaaaggacttctcagccttgctctgaaatgtggtgtttgcttgaccattctgatttttatatcatagatgccaccaagtgcaaacatgtttagaatattataggcattccatttctcagaataaaaaaaaaatgactaattggcttattttcttaagtactcaaaagtatcccatttagctaatgtgtctgagaaatactgcccgtgcatttggtatttctttgattttgtggcactgctgagagtgagagcagaaaggtttttggcagtgtgaattatgctgcgacatgattattatttagatccgtttcataggtgcatgcagtcgttttcttattacagcagtgtaaatgtggcacatttttcatgtgacatagtagctttctaatttatgaagccatgtctgtttacttaggagtatatacattcacacacaaagggtgtgtgtgtttattcacctctcctttcattctttggcacaatggacaacttggtgtataggaaaaaagaaacaaatttggtttctatccacttttttttttaaccagtttttcttgtagttattatttaagctttctttatgttccctgtgttaactatttaagtagcattctttctaaacttacaaaccagacacatttgttgctgtgggtgtgtgcatgggtatatgtgtgtgtgtgtgttctctggagttatgcaaggaagactgttttctttacatatgtgatgatttgcctcattgacaaatttgctctctggttgataaccttcacatccttgtactttttgtatgctcacattttctgggtattatatagagaagcctagaaacactttacatgatgtggtgggatggcatggggttgagatgtgcttctcccctttctgtcctctctggcactctaataattgtgcttttgtttctccaaccacagccgagcctcttgaagccattcttacagatgatgaaccagaccacggcccgttgggagctccagaattcgcggccgcacgcgtcacctgtgggcagtgccagatgaacttcccattgggggacattcttatttttatcgagcacaaacggaaacaatgcaatggcagcctctgcttagaaaaagctgtggataagccaccttccccttcaccaatcgagatgaaaaaagcatccaatcccgtggaggttggcatccaggtcacgccagaggatgacgattgtttatcaacgtcatctagaggaatttgccccaaacaggaacacatagcaggtaaatgagaagcaaggagaaaagctgtttgcatgttttcttttcattttcagaggtgctgtagccaagcagtaaggagttgtgaagtgctttctctattactctatgtgactgtccatgacagccctgtaatgttaaaataatcatttctgttgcttacgtccagaacacagaaaaataaatattttccacctcactgaatcagatgtaggcaggataggtacacacatcagacaccttctctctggatctgtcgattttggatttcttttcttccccatccccaccttctcattttgaagtattgagctttactacacctagtccagcttccattgtccatttccagccttggtgacgtgtcagaggcaaagtggccatataggcatttgcagttcagccaatgacttgtttgactcagaacatctggccaggcctccttaggggttcagctcgttctcaaggcttccctgaagtagagtgggctggcagggtagttggaggtggtggaaagagttaactgagcttcagggctagccttggatccatattggctgtcagcccggatggggctgtaattaaacacagccccgtggtgggatgacaccatgaccttgactttaagatgccattttcgactggccaggccagagtagagagggcagttgctgaagcgcacagacatgcttactcgaaaagtttaagggcatgttggaaatttcaaaaggttggtttgacaggaacggctgctccctgcagcctgcctcctcagctaaatgataaatgcttctctgtgctctctcttgtctctgatgtggttttgacagatgtatcttgattttgtttgtggtttacacagccacatgtcacccttacaaatgtccagtccagactccactgtttctgctataacacaatgtaaaaattttcttggaaaaatacacacacgtattcaacagccctccctcctttggttaattttagcagggaggcagctaggtgtgtgggtttctcggcagctcaagggaaaaggaattaaaggctagcagtgggacttaaattcccttctctaagtgataaacagtaacactatatagtgaccctcaaaacattttttgcttgagcatgttagacaaaagtcaatgcagattctgtgatgacagacatgccatgcctgttggtggatcgctttcttccatctacctaccacccagctcccgaaaggcaagaggtttgttcagttttaggaaaggtagtgcatatcatgaattgattcactggaacttgtctctccgacctagtttgaccacaaagttgaaccataataggtcagtggtctagaggggattaaatgtcatattatttctcctctccccctctagaatttgatcattaaaaccaaacatggcattttctttctttttttagtgctttctgtgatagcactcagatactttccctttagtgaaatgggaaatctgctgctagggaagctgcatttgtggagtgtatttcttgaatccaccacatttaccttatgtgacatgtaggtgaagattttatctcccctaccccccagcaggatgtgggaatgaccatttccatgtgttgtcttgtgactggaaggaaaatgaacagaagtgtaaggcatgattaatgaagcaagagcaggcggaaggggatttgtcgtcttcggagatccaaagccttgctaaatcaccaaatatggagtaacacttgcgtgatgtaacatcgtatttacatatcgagctgctcgtttaaaagacaaaacacagtgtctgtcaagcaagaattaaaaccacacttcttactgaggtcccagaaggggatcatgacctcct

SEQ ID NO: 10 - DNA, 272 bp, Artificial Sequence
  Description of Artificial Sequence Synthetic polynucleotide
atcgatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaattgtgagcgctcacaattcccgttgggagctccagaaggggatcatgacctcctaattgtgagcgctcacaatttaaatagccaccatg

SEQ ID NO: 11 - DNA, 4619 bp, Artificial Sequence
  Description of Artificial Sequence Synthetic polynucleotide
atggataagaagtactcaatcgggctggatatcggaactaattccgtgggttgggcagtgatcacggatgaatacaaagtgccgtccaagaagttcaaggtcctggggaacaccgatagacacagcatcaagaaaaatctcatcggagccctgctgtttgactccggcgaaaccgcagaagcgacccggctcaaacgtaccgcgaggcgacgctacacccggcggaagaatcgcatctgctatctgcaagagatcttttcgaacgaaatggcaaaggtcgacgacagcttcttccaccgcctggaagaatctttcctggtggaggaggacaagaagcatgaacggcatcctatctttggaaacatcgtcgacgaagtggcgtaccacgaaaagtacccgaccatctaccatctgcggaagaagttggttgactcaactgacaaggccgacctcagattgatctacttggccctcgcccatatgatcaaattccgcggacacttcctgatcgaaggcgatctgaaccctgataactccgacgtggataagcttttcattcaactggtgcagacctacaaccaactgttcgaagaaaacccaatcaatgctagcggcgtcgatgccaaggccatcctgtccgcccggctgtcgaagtcgcggcgcctcgaaaacctgatcgcacagctgccgggagagaaaaagaacggacttttcggcaacttgatcgctctctcactgggactcactcccaatttcaagtccaattttgacctggccgaggacgcgaagctgcaactctcaaaggacacctacgacgacgacttggacaatttgctggcacaaattggcgatcagtacgcggatctgttccttgccgctaagaacctttcggacgcaatcttgctgtccgatatcctgcgcgtgaacaccgaaataaccaaagcgccgcttagcgcctcgatgattaagcggtacgacgagcatcaccaggatctcacgctgctcaaagcgctcgtgagacagcaactgcctgaaaagtacaaggagatcttcttcgaccagtccaagaatgggtacgcagggtacatcgatggaggcgctagccaggaagagttctataagttcatcaagccaatcctggaaaagatggacggaaccgaagaactgctggtcaagctgaacagggaggatctgctccggaaacagagaacctttgacaacggatccattccccaccagatccatctgggtgagctgcacgccatcttgcggcgccaggaggacttttacccattcctcaaggacaaccgggaaaagatcgagaaaattctgacgttccgcatcccgtattacgtgggcccactggcgcgcggcaattcgcgcttcgcgtggatgactagaaaatcagaggaaaccatcactccttggaatttcgaggaagttgtggataagggagcttcggcacaaagcttcatcgaacgaatgaccaacttcgacaagaatctcccaaacgagaaggtgcttcctaagcacagcctcctttacgaatacttcactgtctacaacgaactgactaaagtgaaatacgttactgaaggaatgaggaagccggcctttctgtccggagaacagaagaaagcaattgtcgatctgctgttcaagaccaaccgcaaggtgaccgtcaagcagcttaaagaggactacttcaagaagatcgagtgtttcgactcagtggaaatcagcggggtggaggacagattcaacgcttcgctgggaacctatcatgatctcctgaagatcatcaaggacaaggacttccttgacaacgaggagaacgaggacatcctggaagatatcgtcctgaccttgacccttttcgaggatcgcgagatgatcgaggagaggcttaagacctacgctcatctcttcgacgataaggtcatgaaacaactcaagcgccgccggtacactggttggggccgcctctcccgcaagctgatcaacggtattcgcgataaacagagcggtaaaactatcctggatttcctcaaatcggatggcttcgctaatcgtaacttcatgcaattgatccacgacgacagcctgacctttaaggaggacatccaaaaagcacaagtgtccggacagggagactcactccatgaacacatcgcgaatctggccggttcgccggcgattaagaagggaattctgcaaactgtgaaggtggtcgacgagctggtgaaggtcatgggacggcacaaaccggagaatatcgtgattgaaatggcccgagaaaaccagactacccagaagggccagaaaaactcccgcgaaaggatgaagcggatcgaagaaggaatcaaggagctgggcagccagatcctgaaagagcacccggtggaaaacacgcagctgcagaacgagaagctctacctgtactatttgcaaaatggacgggacatgtacgtggaccaagagctggacatcaatcggttgtctgattacgacgtggaccacatcgttccacagtcctttctgaaggatgactcgatcgataacaaggtgttgactcgcagcgacaagaacagagggaagtcagataatgtgccatcggaggaggtcgtgaagaagatgaagaattactggcggcagctcctgaatgcgaagctgattacccagagaaagtttgacaatctcactaaagccgagcgcggcggactctcagagctggataaggctggattcatcaaacggcagctggtcgagactcggcagattaccaagcacgtggcgcagatcttggactcccgcatgaacactaaatacgacgagaacgataagctcatccgggaagtgaaggtgattaccctgaaaagcaaacttgtgtcggactttcggaaggactttcagttttacaaagtgagagaaatcaacaactaccatcacgcgcatgacgcatacctcaacgctgtggtcggtaccgccctgatcaaaaagtaccctaaacttgaatcggagtttgtgtacggagactacaaggtctacgacgtgaggaagatgatagccaagtccgaacaggaaatcgggaaagcaactgcgaaatacttcttttactcaaacatcatgaactttttcaagactgaaattacgctggccaatggagaaatcaggaagaggccactgatcgaaactaacggagaaacgggcgaaatcgtgtgggacaagggcagggacttcgcaactgttcgcaaagtgctctctatgccgcaagtcaatattgtgaagaaaaccgaagtgcaaaccggcggattttcaaaggaatcgatcctcccaaagagaaatagcgacaagctcattgcacgcaagaaagactgggacccgaagaagtacggaggattcgattcgccgactgtcgcatactccgtcctcgtggtggccaaggtggagaagggaaagagcaaaaagctcaaatccgtcaaagagctgctggggattaccatcatggaacgatcctcgttcgagaagaacccgattgatttcctcgaggcgaagggttacaaggaggtgaagaaggatctgatcatcaaactccccaagtactcactgttcgaactggaaaatggtcggaagcgcatgctggcttcggccggagaactccaaaaaggaaatgagctggccttgcctagcaagtacgtcaacttcctctatcttgcttcgcactacgaaaaactcaaagggtcaccggaagataacgaacagaagcagcttttcgtggagcagcacaagcattatctggatgaaatcatcgaacaaatctccgagttttcaaagcgcgtgatcctcgccgacgccaacctcgacaaagtcctgtcggcctacaataagcatagagataagccgatcagagaacaggccgagaacattatccacttgttcaccctgactaacctgggagccccagccgccttcaagtacttcgatactactatcgatcgcaaaagatacacgtccaccaaggaagttctggacgcgaccctgatccaccaaagcatcactggactctacgaaactaggatcgatctgtcgcagctgggtggcgatggctcggcttacccatacgacgtgcctgactacgcctcgctcggatcgggctcccccaaaaagaaacggaaggtggacggatccccgaaaaagaagagaaaggtggactccggatgagaattctcacggctttccgcctgaggttgaagagcaagccgccggtacattgcctatgtcctgcgcacaagaaagcggtatggaccggcacccagccgcttgtgcttcagctcgcatcaacgtctaaggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataa

SEQ ID NO: 12 - DNA, 4344 bp, Artificial Sequence
  Description of Artificial Sequence Synthetic polynucleotide
atggataagaagtactcaatcgggctggatatcggaactaattccgtgggttgggcagtgatcacggatgaatacaaagtgccgtccaagaagttcaaggtcctggggaacaccgatagacacagcatcaagaaaaatctcatcggagccctgctgtttgactccggcgaaaccgcagaagcgacccggctcaaacgtaccgcgaggcgacgctacacccggcggaagaatcgcatctgctatctgcaagagatcttttcgaacgaaatggcaaaggtcgacgacagcttcttccaccgcctggaagaatctttcctggtggaggaggacaagaagcatgaacggcatcctatctttggaaacatcgtcgacgaagtggcgtaccacgaaaagtacccgaccatctaccatctgcggaagaagttggttgactcaactgacaaggccgacctcagattgatctacttggccctcgcccatatgatcaaattccgcggacacttcctgatcgaaggcgatctgaaccctgataactccgacgtggataagcttttcattcaactggtgcagacctacaaccaactgttcgaagaaaacccaatcaatgctagcggcgtcgatgccaaggccatcctgtccgcccggctgtcgaagtcgcggcgcctcgaaaacctgatcgcacagctgccgggagagaaaaagaacggacttttcggcaacttgatcgctctctcactgggactcactcccaatttcaagtccaattttgacctggccgaggacgcgaagctgcaactctcaaaggacacctacgacgacgacttggacaatttgctggcacaaattggcgatcagtacgcggatctgttccttgccgctaagaacctttcggacgcaatcttgctgtccgatatcctgcgcgtgaacaccgaaataaccaaagcgccgcttagcgcctcgatgattaagcggtacgacgagcatcaccaggatctcacgctgctcaaagcgctcgtgagacagcaactgcctgaaaagtacaaggagatcttcttcgaccagtccaagaatgggtacgcagggtacatcgatggaggcgctagccaggaagagttctataagttcatcaagccaatcctggaaaagatggacggaaccgaagaactgctggtcaagctgaacagggaggatctgctccggaaacagagaacctttgacaacggatccattccccaccagatccatctgggtgagctgcacgccatcttgcggcgccaggaggacttttacccattcctcaaggacaaccgggaaaagatcgagaaaattctgacgttccgcatcccgtattacgtgggcccactggcgcgcggcaattcgcgcttcgcgtggatgactagaaaatcagaggaaaccatcactccttggaatttcgaggaagttgtggataagggagcttcggcacaaagcttcatcgaacgaatgaccaacttcgacaagaatctcccaaacgagaaggtgcttcctaagcacagcctcctttacgaatacttcactgtctacaacgaactgactaaagtgaaatacgttactgaaggaatgaggaagccggcctttctgtccggagaacagaagaaagcaattgtcgatctgctgttcaagaccaaccgcaaggtgaccgtcaagcagcttaaagaggactacttcaagaagatcgagtgtttcgactcagtggaaatcagcggggtggaggacagattcaacgcttcgctgggaacctatcatgatctcctgaagatcatcaaggacaaggacttccttgacaacgaggagaacgaggacatcctggaagatatcgtcctgaccttgacccttttcgaggatcgcgagatgatcgaggagaggcttaagacctacgctcatctcttcgacgataaggtcatgaaacaactcaagcgccgccggtacactggttggggccgcctctcccgcaagctgatcaacggtattcgcgataaacagagcggtaaaactatcctggatttcctcaaatcggatggcttcgctaatcgtaacttcatgcaattgatccacgacgacagcctgacctttaaggaggacatccaaaaagcacaagtgtccggacagggagactcactccatgaacacatcgcgaatctggccggttcgccggcgattaagaagggaattctgcaaactgtgaaggtggtcgacgagctggtgaaggtcatgggacggcacaaaccggagaatatcgtgattgaaatggcccgagaaaaccagactacccagaagggccagaaaaactcccgcgaaaggatgaagcggatcgaagaaggaatcaaggagctgggcagccagatcctgaaagagcacccggtggaaaacacgcagctgcagaacgagaagctctacctgtactatttgcaaaatggacgggacatgtacgtggaccaagagctggacatcaatcggttgtctgattacgacgtggaccacatcgttccacagtcctttctgaaggatgactcgatcgataacaaggtgttgactcgcagcgacaagaacagagggaagtcagataatgtgccatcggaggaggtcgtgaagaagatgaagaattactggcggcagctcctgaatgcgaagctgattacccagagaaagtttgacaatctcactaaagccgagcgcggcggactctcagagctggataaggctggattcatcaaacggcagctggtcgagactcggcagattaccaagcacgtggcgcagatcttggactcccgcatgaacactaaatacgacgagaacgataagctcatccgggaagtgaaggtgattaccctgaaaagcaaacttgtgtcggactttcggaaggactttcagttttacaaagtgagagaaatcaacaactaccatcacgcgcatgacgcatacctcaacgctgtggtcggtaccgccctgatcaaaaagtaccctaaacttgaatcggagtttgtgtacggagactacaaggtctacgacgtgaggaagatgatagccaagtccgaacaggaaatcgggaaagcaactgcgaaatacttcttttactcaaacatcatgaactttttcaagactgaaattacgctggccaatggagaaatcaggaagaggccactgatcgaaactaacggagaaacgggcgaaatcgtgtgggacaagggcagggacttcgcaactgttcgcaaagtgctctctatgccgcaagtcaatattgtgaagaaaaccgaagtgcaaaccggcggattttcaaaggaatcgatcctcccaaagagaaatagcgacaagctcattgcacgcaagaaagactgggacccgaagaagtacggaggattcgattcgccgactgtcgcatactccgtcctcgtggtggccaaggtggagaagggaaagagcaaaaagctcaaatccgtcaaagagctgctggggattaccatcatggaacgatcctcgttcgagaagaacccgattgatttcctcgaggcgaagggttacaaggaggtgaagaaggatctgatcatcaaactccccaagtactcactgttcgaactggaaaatggtcggaagcgcatgctggcttcggccggagaactccaaaaaggaaatgagctggccttgcctagcaagtacgtcaacttcctctatcttgcttcgcactacgaaaaactcaaagggtcaccggaagataacgaacagaagcagcttttcgtggagcagcacaagcattatctggatgaaatcatcgaacaaatctccgagttttcaaagcgcgtgatcctcgccgacgccaacctcgacaaagtcctgtcggcctacaataagcatagagataagccgatcagagaacaggccgagaacattatccacttgttcaccctgactaacctgggagccccagccgccttcaagtacttcgatactactatcgatcgcaaaagatacacgtccaccaaggaagttctggacgcgaccctgatccaccaaagcatcactggactctacgaaactaggatcgatctgtcgcagctgggtggcgatggctcggcttacccatacgacgtgcctgactacgcctcgctcggatcgggctcccccaaaaagaaacggaaggtggacggatccccgaaaaagaagagaaaggtggactccgggaattctcacggctttccgcctgaggttgaagagcaagccgccggtacattgcctatgtcctgcgcacaagaaagcggtatggaccggcacccagccgcttgtgcttcagctcgcatcaacgtctaa

SEQ ID NO: 13 - DNA, 351 bp, Artificial Sequence
  Description of Artificial Sequence Synthetic polynucleotide
gggcctattttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaggaggtcatgatccccttcgttttagagctagaaatagcaagttaaaataaggctagtccgttatcaacttgaaaaagtggcaccgagtcggtgcttttttt

SEQ ID NO: 14 - DNA, 1470 bp, Artificial Sequence
  Description of Artificial Sequence Synthetic polynucleotide
atgaagcctgtgaccctgtacgacgtggccgagtacgccggagtgagttatcagactgtgtcccgggtcgtgaatcaggcctctcacgtgagcgctaaaacccgcgagaaagtggaggccgcaatggccgaactgaactatatcccaaaccgcgtggcccagcagctggcaggcaagcagagcctgctgatcggcgtggctacctctagcctcgcattgcacgccccgtctcagatcgtggccgccatcaagtcccgcgctgatcagctgggagcttctgtcgtggtgagcatggtcgaacgctccggagtggaggcctgcaaggcagccgtccataacctcctcgcccagcgggtttccggcctgattatcaactatccactggatgaccaagatgccatcgctgtcgaggcggcatgtactaacgtgccagccttgtttctggatgtgagcgaccagacccctatcaacagcatcatcttctctcacgaggacggcactaggctgggtgttgagcacctggtcgccctgggtcaccagcagattgccctcttggccggcccactgtctagtgtgagtgcgagactgcgtttggccggttggcacaagtacctgacacgcaaccagatccagcccatcgcggagcgtgagggagactggagtgccatgtccggcttccagcagaccatgcagatgctgaacgaaggaattgtgccaaccgccatgctcgtggcaaatgatcagatggccctgggagccatgagggcaatcactgagtctggcctccgtgtgggtgccgatatcagtgttgtgggctacgacgatactgaggactctagttgttatatccctcccctcaccaccatcaaacaggacttcaggctcctgggccaaacctccgtcgaccggttgctgcagctcagccagggccaggccgtcaagggaaaccagctcctgccagtttccttggtgaagcgtaaaacaaccctggcaccaaatactcagacagcgtctcctcgcgcgctcgcggattctctgatgcagctcgcacgtcaggtgagcaggttggagtccggccaaccgaagaagaagagaaaggtcgacggaggtggcgctctgtcccctcagcacagtgctgtgacacagggcagcatcatcaagaacaaggagggcatggacgccaagagcctcactgcctggtcccgtaccctggtgactttcaaggacgtgttcgttgactttacccgggaggagtggaagctgctggataccgcccagcagattgtctatagaaacgtcatgctggagaactacaagaatctcgtgtccctcggataccagctgacaaaacctgacgtgatcctgcgcctcgagaagggtgaagaaccttggctggtggaaagggaaattcaccaggagacccaccccgattccgagaccgccttcgagatcaaaagcagtgtgtaatga

SEQ ID NO: 15 - DNA, 4988 bp, Artificial Sequence
  Description of Artificial Sequence Synthetic polynucleotide
gctgccaggaacctacattgtgggaggaataactctatccatcaaagtaatgccctgggcaagatgcttcctctccccctttagcagtgaagtgtaggcactgaaggccattatatcatcaccctttcaggcctagaaatctttttcggctctaacatagcagagccatttgattcactgtctgatggtcataacacatttgcctctcaaaccctatcttctgtcctaacccccaagctgctcagcactggttaccatcggaaggtttggcattttgattttatgctgtttgattacagtctctttatgccatcgagccctgaactgaagggatttagcaggttttaaacaagtcctggccagcgtgtcccactcatggggtattaggtggtctgcttcagccgtccctttcaacaattccaaagccatatggagatatagcttcagaagagggcatggcatgtttaaaacccccaagtgtcgtatagggaagggaacaggctcatcctctgtgtgtattcctcactgaggaaaagcatcgtcaactcttcgtgatggtggtggattcaaggattgaaggggatggaaatacaagaggcaaggaggttaggcatgtctcaggatccttctttttgagctaacagaacctcccaggataatgcaaatgcatcagcccgtaggggtgcagaggaagggctagtagggtgcagaggaagggctagtaggggtgcagaggaagggctagtagggtgcagaggaagggctagtagaggtgcagaggaagggctagtaggggtgcagaggaagggctagtagggtgcagaggaagggctagtagggtgcagaggaagggctagtaggggtgcagaggaagggctagtaggggtgcagagaaagggctagtagggtgcagaggaagggctagtagggtgcagaggaagggctagtaggggtgcagaggaagggctagtagggtgcagaggaagggctagtagggtgcagaggaagggctagtagggtgcagaggaagggctagtaggggtgcagaggaagggctagtagggtgcagagaaagggctagtagggtgcagaggaagggctagtagggtgcagaggaagggctagtaggggtgcagaggaagggctagtaaagtgcagaggaagggctagtagaggatgctctgtcttctgaatcattggaagaatcagaagactgggaatggggtgaggggagctgaaggcttcaggcaaggcttgcctacttctgtctctctgaagggtctatctggtgctttctctctgtgcttagggtaggggtggtttgcaaagcctgaatagctaaggtgatcagattaaaaggggctggacattgaatgggcccacctctccccgcccatgaacttgtttaaaataacacaaaacacctttccattgctttatgtgtaatgtgccctatggtggcagtcaggagcagtatgtccatgtattctgacaggctatagagatctgctttttgccccttccaccatgctttgacccctctgcacaataggcacattgtagtcttttcttttgttttgctttgcacccatgattaccctggtgtcctggtgtgggctcccatgtgtgtaccaggactacatacctctcattagattccctctgttttgctcaggccctgtttgggtaccacacgtttcaatccaccaatgattggtgcaactcaagattcaacaaggccagggcctctatgctccataagaacctttttattggagttctgtggagagtttatttggatagttcagggttcaaagcatgggcagagaaacagtgaaaaatatacacattatttatgattattctcaccagatgtactcaggagacagaaggttctacaggaacagagtgcatgcaacaagacaacatgggaaaatctgtgatacgcatgctacactgagatgaggtcatgctggggtcctcacgttctctgcttctcttccttcttggggatcaggaggcctggggatcttccggagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgaattctcacggctttccgcctgaggttgaagagcaagccgccggtacattgcctatgtcctgcgcacaagaaagcggtatggaccggcacccagccgcttgtgcttcagctcgcatcaacgtctaaggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaggcgcgcctgtggtgctgatggaggagacccagaggctacagattgaacaaactgcccaccgcctgcagacccgggctgcccgccggggctatgtcatcaaggttctacatatcttttatgacctcttccctggcttcttggtgaagatgagcagtgacctgttgggcctggtgagccatcttcttgggcgtgggactttccaggaaggatggacttccatgtccatgtctcgactgaccttagtgtgcccactgccgagaggcaggaccagtaagcgcctgtggctcttggttccctgaataactaactgcctacttaacttggccacatccccatgctgtgtctacaatcataggaggacagaggggatcacaaggcagctagcagcagagcccctgcctgccagtatacgtttctggtttgtctactgcctgtgaaaacctgcagggacaaggcctggggatgctggtgacaaaggtgtcaaatatgtcagattcttctcgtttgggacaaggtagtgcttctccataactcctctgaatgttgcctttctttgctaagaaggtaaaaggggtacagactatcacctgccctccctgtcttctccccatcttggcactccaggttctcacctctcttctccactgggtactctcagccccctgcacacccttaggtcccagggctcagggccttgcccaggcctccagtgtgcattgcacatgcacctggtcttctggcctcaggttcctgctcagggtttaaactgacttaagatcttgttactaaatgacagtggggcatgggccatgccacggagcaggaggacatcaaatcagtgcctcccatccatgcactctgcactttaccaagcatcgcctgtgacacagccttgaacctttccatcaagcttacggcaaaggtggagactggatggatggttgatgccagagcagttagttggtgctgtgtgctcagtgcagtggggaatgagtaagacacctgaagtgccagggggccggcaggtgcccgctggtcagggcaacaagctctgtacagggggccataggatttgctctaggaacttgagcccggagtctcaaagggtgcactggcctcagctcagtgtcccactagtttgtttagtttagagcagatgccactctctcccacgattatccgtaagccagatggggtgatgggagccatcttttgaggactagtggagactgtggaatatttttttgaatagggataggttgagacagtgtcttgttttgtagtccaagctggcctctcactcgtgataaccccacccctgcttctgggatgcttgtattacagacaggtgccaacatcaccagctgaatgttgagcgtttaagcagatattaagtaaagacactggcagagggtaggagtcctggggatactgaagcacctagagatgtcttgggcctctaggagtggggtgaagagaggaaactgaagcatggaggaagggcgtggtatctgaggatgtagatgtgtaagcctggctaaggagcagggtgcaggcccctctctcaaactaatgcagatgcctcctattagccaaacacactggaggctgggaggctggttgctgtggtctgcagggccagtgcaaaggccagggatgggagcagaggccccatggccagcactggtatcctgactggagattgacggtactaagattcctgaccacatccctgaagctaggcataacctgactctcaggggagatgtggagctcagaatccagagagtggaatagagaaccctccgagcaggcatatagattcaggggctggagttacggaacaccgtgctccccagccagagagaaatgaggacactggcccctggtctgtcttctgggccccaggaggaagactttgtgaaggctggggaggtggacagtcaggtggggctgctgtgggctgctattagctgaagggcttttgaagctaagtgcatggctgtctggttctgtaggccctgaagttccacaatgtaggttcctggcagc

SEQ ID NO: 16 - DNA, 365 bp, Artificial Sequence
  Description of Artificial Sequence Synthetic polynucleotide
gaattgatactcgagggcctattttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgccaggaacctacattggttttagagctagaaatagcaagttaaaataaggctagtccgttatcaacttgaaaaagtggcaccgagtcggtgcttttttt

(19) United States
(12) Patent Application Publication
(10) Pub. No.: US 2018/0112234 A9
(43) Pub. Date: Apr. 26, 2018

(54) METHODS AND COMPOSITIONS FOR GENE EDITING

(71) Applicant: Intellia Therapeutics, Inc., Cambridge, MA (US)

(72) Inventors: Christian Dombrowski, Boston, MA (US); Thomas Michael Barnes, Brookline, MA (US)

(73) Assignee: Intellia Therapeutics, Inc.

(21) Appl. No.: 15/457,866

(22) Filed: Mar. 13, 2017

Related U.S. Application Data
(60) Provisional application No. 62/308,032, filed on Mar. 14, 2016.

(51) Int. Cl. C12N 15/90; C12N 9/22; C12N 15/11; C12N 15/86; C12N 7/00

(52) U.S. Cl. CPC C12N 15/907; C12N 9/22; C12N 15/11; C12N 2830/005; C12N 7/00; C12N 2310/20; C12N 15/86

(57) ABSTRACT

Compositions and methods are provided for enhancing the efficiency of gene editing by timing the expression and activity of a nuclease to correspond with availability of a repair template. Compositions and methods for temporally regulating the duration of nuclease activity, and methods of selectively preventing nuclease expression during viral vector production, are also provided.

The present application claims the benefit of priority to U.S. Provisional Patent Application No. 62/308,032, filed Mar. 14, 2016, which is incorporated herein by reference.

The instant application contains a Sequence Listing which has been submitted electronically in ASCII format and is hereby incorporated by reference in its entirety. The ASCII copy, created on Mar. 13, 2017, is named 12793_0003-00000_SL.txt and is 30,381 bytes in size.

A number of methods for editing genes in cells in vivo now exist, providing tremendous potential for treating genetic, viral, and bacterial diseases. Several of these editing technologies take advantage of cellular mechanisms for repairing double-stranded breaks (“DSB”) created by enzymes such as meganucleases, clustered regularly interspaced short palindromic repeats (CRISPR) associated nucleases (“Cas”), zinc finger nucleases (“ZFN”), and transcription activator-like effector nucleases (“TALEN”). In certain circumstances, cells repair DSBs by homology-directed repair (“HDR”) or homologous recombination (“HR”) mechanisms, where an endogenous or exogenous template with homology to each end of a DSB is used to direct repair of the break.

The efficiencies of HDR and HR mechanisms may be correlated with the availability of the repair template at or near the site of the DSB. One method for performing gene editing in vivo involves delivering a DSB-generating enzyme along with a repair template via a viral vector. Using such methods, it can be difficult to successfully edit genes via HDR and HR because the expression and activity of the enzyme is not optimally timed with the presence of the repair template. We herein describe compositions and methods for enhancing the efficiency of gene editing via HDR and HR by timing the expression and activity of a DSB-generating enzyme to correspond with availability of a repair template, by liberating that template from the recombinant viral vector via vector cleavage.

Additionally, we provide compositions and methods for temporally regulating the duration of enzyme activity to improve gene editing results, including self-regulation of enzyme expression via vector cleavage.

In embodiments where the enzyme cleaves the recombinant viral vector, manufacturing such a vector in a cell system may pose significant challenges. Accordingly, we describe methods of selectively preventing enzyme expression such that the vector can be successfully produced and packaged into a viral delivery system.

### SUMMARY

A vector system is provided, which may comprise one or more vectors encoding: 1) a nuclease system that cleaves a first target sequence on a target nucleic acid molecule, the nuclease system comprising at least one nuclease, wherein the vector encoding the nuclease comprises a nucleotide sequence encoding the nuclease operably linked to a first promoter, and a second target sequence that the nuclease system cleaves and reduces the expression of at least one component of the nuclease system; and 2) a template sequence flanked at each end respectively by a third target sequence and a fourth target sequence that the nuclease system cleaves.

In another aspect, a method for editing a target nucleic acid molecule in a eukaryotic cell is provided, the method comprising administering the vector system described herein.

Embodiments also include a method for producing a virus comprising a nucleic acid, the method comprising: providing a cell expressing a LacI protein; introducing into the cell the nucleic acid; introducing into the cell one or more viral components for producing the virus; growing the cell; and isolating the virus comprising a nucleic acid from the cell, wherein the nucleic acid encodes: 1) a nuclease system that cleaves a first target sequence on a target nucleic acid molecule, the nuclease system comprising at least one nuclease, wherein the nucleic acid comprises: a nucleotide sequence encoding the nuclease operably linked to a first promoter, a second target sequence that the nuclease system cleaves and reduces the expression of at least one component of the nuclease system, and at least two lacO sequences within the first promoter or between the first promoter and the nucleotide sequence encoding the nuclease, and 2) a template sequence flanked at each end respectively by a third target sequence and a fourth target sequence that the nuclease system cleaves.

Embodiments also encompass a method for producing a virus comprising a nucleic acid, the method comprising: introducing into a cell a vector comprising a nucleotide sequence encoding a LacI protein, the nucleic acid, and one or more viral components for producing the virus; growing the cell; and isolating the virus comprising a nucleic acid from the cell, wherein the nucleic acid encodes: 1) a nuclease system that cleaves a first target sequence on a target nucleic acid molecule, the nuclease system comprising at least one nuclease, wherein the nucleic acid comprises: a nucleotide sequence encoding the nuclease operably linked to a first promoter, a second target sequence that the nuclease system cleaves and reduces the expression of at least one component of the nuclease system, and at least two lacO sequences within the first promoter or between the first promoter and the nucleotide sequence encoding the nuclease, and 2) a template sequence flanked at each end respectively by a third target sequence and a fourth target sequence that the nuclease system cleaves.

Further provided is a self-regulating vector encoding: 1) a CRISPR/Cas9 system that cleaves a target sequence on a target nucleic acid molecule, the CRISPR/Cas9 system comprising a Cas9 protein and a guide RNA, wherein the vector comprises (i) a nucleotide sequence encoding the Cas9 protein operably linked to a first promoter, (ii) a nucleotide sequence encoding the guide RNA operably linked to a second promoter, and (iii) the target sequence which reduces the expression of the Cas9 protein or the guide RNA; and 2) a template sequence flanked at each end by the target sequence.

### BRIEF DESCRIPTION OF DRAWINGS

FIG. 1 shows an exemplary vector containing sequences encoding a CRISPR/Cas9 nuclease system, a template sequence, and target sequences for the nuclease. The vector includes sequences encoding the Cas9 enzyme, a guide RNA sequence, and a template, as well as target sequences placed such that the Cas9/guide RNA combination cleaves the vector to release the template and simultaneously but independently reduce Cas9 expression. To prevent expression of Cas9 during vector production, the vector also includes lacO elements in the promoter region for the Cas9 sequence.

FIG. 2 shows luciferase activity expressed from a plasmid with a CRISPR/Cas9 cleavage site after incubation for 24 or 44 hours with various amounts of plasmids expressing Cas9 and/or guide RNA. Higher luciferase activity indicates lower amounts of cleavage by CRISPR/Cas9.

FIG. 3 shows cleavage of a plasmid containing a template sequence flanked by target sequences for guide RNA G5 and ClaI/XhoI. The top of the figure is a diagram of the plasmid construct, and the bottom shows cleavage products resulting from a ClaI/XhoI digest on the left, and Cas9/guide RNA G5 on the right (middle lane is size marker).

FIGS. 4A and 4B show homologous recombination of a template released by a vector system that co-expresses Cas9 and guide RNA sequences. The template contains an EcoRI restriction site not present in the wild-type genomic sequence. FIG. 4A is a diagram showing the template and the position of PCR primers (arrows) used for detecting the recombination product, and restriction enzyme cleavage sites. The amplified recombination product will generate 77 bp, 823 bp, and 1349 bp fragments upon cleavage by EcoRI and BamHI, while the wild-type sequence will generate 900 bp and 1349 bp fragments. FIG. 4B shows the fragment analysis for cells transfected with varying amounts of plasmids expressing Cas9 and/or guide RNA sequences.

FIGS. 5A and 5B show homologous recombination products for cells transfected with plasmids expressing guide RNA, template, and various Cas9 constructs containing sequences and/or tags for modulating Cas9 DNA, mRNA, and protein half-life. FIGS. 5A and 5B show results at 24 and 48 hours after transfection, respectively.

FIGS. 6A and 6B show homologous recombination products for cells transfected with plasmids expressing guide RNA, template, and various Cas9 constructs containing sequences and/or tags for modulating Cas9 DNA, mRNA, and protein half-life. FIG. 6A shows results at 24 hours after transfection. FIG. 6B shows results using primers only found in genomic DNA.

FIG. 7 shows luciferase expression from a construct containing lacO sequences inserted between the promoter sequence and the luciferase sequence, in the presence or absence of a plasmid expressing LacI-KRAB fusion protein.

FIG. 8A depicts a schematic of an HR template that was designed for integrating a luciferase reporter gene (Nluc) into the mouse PCSK9 gene. In some embodiments, the HR template does not have a promoter for expressing Nluc and the ATG transcriptional start site is removed from the Nluc coding sequence. Thus, Nluc is expressed from the template if HR occurs between the template and the genomic PCSK9 gene, thereby inserting the Nluc sequence in-frame with the PCSK9 signal peptide, leading to secretion of the Nluc reporter gene into the culture media. The cr437 guide RNA targets a specific sequence in the mouse PCSK9 gene. FIG. 8B depicts an expected HR product wherein the template is inserted in-frame into the PCSK9 gene.

FIG. 9 shows luciferase activity using Plasmids C, D, and/or E. Samples without Plasmid C (i.e., no Cas9) or without Plasmid D or Plasmid E (i.e., no template) showed no luciferase activity in the media at 72 hours post-transfection. Samples with any amount of Cas9 (from Plasmid C) and any amount of template (from Plasmid D or Plasmid E) showed significant luciferase activity, indicating that guide RNA and Cas9 produced from Plasmids C and D/E successfully cleaved the PCSK9 target sequence, resulting in HR and the in-frame insertion of Nluc into PCSK9.

### DETAILED DESCRIPTION

Nuclease Systems

In some embodiments of the present disclosure, the nuclease system includes at least one nuclease. In some embodiments, the nuclease may comprise at least one DNA binding domain and at least one nuclease domain. In some embodiments, the nuclease domain may be heterologous to the DNA binding domain. In certain embodiments, the nuclease is a DNA endonuclease, and may cleave single or double-stranded DNA. In certain embodiments, the nuclease may cleave RNA.

(a) CRISPR/Cas Nuclease System

(1) Cas Nuclease

In some embodiments, the nuclease may include a Cas protein (also called a “Cas nuclease”) from a CRISPR/Cas system. The Cas protein may comprise at least one domain that interacts with a guide RNA (gRNA). Additionally, the Cas protein may be directed to a target sequence by a guide RNA. The guide RNA interacts with the Cas protein as well as the target sequence such that, once directed to the target sequence, the Cas protein is capable of cleaving the target sequence. In certain embodiments, e.g., Cas9, the Cas protein is a single-protein effector, an RNA-guided nuclease. In some embodiments, the guide RNA provides the specificity for the targeted cleavage, and the Cas protein may be universal and paired with different guide RNAs to cleave different target sequences. The terms Cas protein and Cas nuclease are used interchangeably herein.

In some embodiments, the CRISPR/Cas system may comprise Type-I, Type-II, or Type-III system components. Updated classification schemes for CRISPR/Cas loci define Class 1 and Class 2 CRISPR/Cas systems, having Types I to V or VI. See, e.g., Makarova et al., Nat Rev Microbiol, 13(11): 722-36 (2015); Shmakov et al., Molecular Cell, 60:385-397 (2015). Class 2 CRISPR/Cas systems have single protein effectors. Cas proteins of Types II, V, and VI may be single-protein, RNA-guided endonucleases, herein called “Class 2 Cas nucleases.” Class 2 Cas nucleases include, for example, Cas9, Cpf1, C2c1, C2c2, and C2c3 proteins. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables 51 and S3.

In some embodiments, the Cas protein may be from a Type-II CRISPR/Cas system, i.e., a Cas9 protein from a CRISPR/Cas9 system. In some embodiments, the Cas protein may be from a Class 2 CRISPR/Cas system, i.e., a single-protein Cas nuclease such as a Cas9 protein or a Cpf1 protein. The Cas9 and Cpf1 family of proteins are enzymes with DNA endonuclease activity, and they can be directed to cleave a desired nucleic acid target by designing an appropriate guide RNA, as described further herein.

A Type-II CRISPR/Cas system component may be from a Type-IIA, Type-IIB, or Type-IIC system. Cas9 and its orthologs are encompassed. Non-limiting exemplary species that the Cas9 protein or other components may be from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, or Acaryochloris marina. In some embodiments, the Cas9 protein may be from Streptococcus pyogenes. In some embodiments, the Cas9 protein may be from Streptococcus thermophilus. In some embodiments, the Cas9 protein may be from Neisseria meningitidis. In some embodiments, the Cas9 protein may be from Staphylococcus aureus.

In some embodiments, a Cas protein may comprise more than one nuclease domain. For example, a Cas9 protein may comprise at least one RuvC-like nuclease domain (e.g. Cpf1) and at least one HNH-like nuclease domain (e.g. Cas9). In some embodiments, the Cas9 protein may be capable of introducing a DSB in the target sequence. In some embodiments, the Cas9 protein may be modified to contain only one functional nuclease domain. For example, the Cas9 protein may be modified such that one of the nuclease domains is mutated or fully or partially deleted to reduce its nucleic acid cleavage activity. In some embodiments, the Cas9 protein may be modified to contain no functional RuvC-like nuclease domain. In other embodiments, the Cas9 protein may be modified to contain no functional HNH-like nuclease domain. In some embodiments in which only one of the nuclease domains is functional, the Cas9 protein may be a nickase that is capable of introducing a single-stranded break (a “nick”) into the target sequence. In some embodiments, a conserved amino acid within a Cas9 protein nuclease domain is substituted to reduce or alter a nuclease activity. In some embodiments, the Cas protein nickase may comprise an amino acid substitution in the RuvC-like nuclease domain. Exemplary amino acid substitutions in the RuvC-like nuclease domain include D10A (based on the S. pyogenes Cas9 protein). In some embodiments, the nickase may comprise an amino acid substitution in the HNH-like nuclease domain. Exemplary amino acid substitutions in the HNH-like nuclease domain include E762A, H840A, N863A, H983A, and D986A (based on the S. pyogenes Cas9 protein). In some embodiments, the nuclease system described herein may comprise a nickase and a pair of guide RNAs that are complementary to the sense and antisense strands of the target sequence, respectively. The guide RNAs may direct the nickase to target and introduce a DSB by generating a nick on opposite strands of the target sequence (i.e., double nicking). Chimeric Cas9 proteins may also be used, where one domain or region of the protein is replaced by a portion of a different protein. For example, a Cas9 nuclease domain may be replaced with a domain from a different nuclease such as Fok1. A Cas9 protein may be a modified nuclease.

In alternative embodiments, the Cas protein may be from a Type-I CRISPR/Cas system. In some embodiments, the Cas protein may be a component of the Cascade complex of a Type-I CRISPR/Cas system. For example, the Cas protein may be a Cas3 protein. In some embodiments, the Cas protein may be from a Type-III CRISPR/Cas system. In some embodiments, the Cas protein may be from a Type-IV CRISPR/Cas system. In some embodiments, the Cas protein may be from a Type-V CRISPR/Cas system. In some embodiments, the Cas protein may be from a Type-VI CRISPR/Cas system. In some embodiments, the Cas protein may have an RNA cleavage activity.

(2) Guide RNA

In some embodiments of the present disclosure, a CRISPR/Cas nuclease system includes at least one guide RNA. In some embodiments, the guide RNA and the Cas protein may form a ribonucleoprotein (RNP), e.g., a CRISPR/Cas complex. The guide RNA may guide the Cas protein to a target sequence on a target nucleic acid molecule, where the guide RNA hybridizes with and the Cas protein cleaves the target sequence. In some embodiments, the CRISPR/Cas complex may be a Cpf1/guide RNA complex. In some embodiments, the CRISPR complex may be a Type-II CRISPR/Cas9 complex. In some embodiments, the Cas protein may be a Cas9 protein. In some embodiments, the CRISPR/Cas9 complex may be a Cas9/guide RNA complex.

A guide RNA for a CRISPR/Cas9 nuclease system comprises a CRISPR RNA (crRNA) and a tracr RNA (tracr). A guide RNA for a CRISPR/Cpf1 nuclease system comprises a crRNA. In some embodiments, the crRNA may comprise a targeting sequence that is complementary to and hybridizes with the target sequence on the target nucleic acid molecule. The crRNA may also comprise a flagpole that is complementary to and hybridizes with a portion of the tracrRNA. In some embodiments, the crRNA may parallel the structure of a naturally occurring crRNA transcribed from a CRISPR locus of a bacteria, where the targeting sequence acts as the spacer of the CRISPR/Cas9 system, and the flagpole corresponds to a portion of a repeat sequence flanking the spacers on the CRISPR locus.

The guide RNA may target any sequence of interest via the targeting sequence of the crRNA. In some embodiments, the degree of complementarity between the targeting sequence of the guide RNA and the target sequence on the target nucleic acid molecule may be about 60%, 65%, 70%, 75%, 80%, 85%, 90%, 95%, 97%, 98%, 99%, or 100%. In some embodiments, the targeting sequence of the guide RNA and the target sequence on the target nucleic acid molecule may be 100% complementary. In other embodiments, the targeting sequence of the guide RNA and the target sequence on the target nucleic acid molecule may contain at least one mismatch. For example, the targeting sequence of the guide RNA and the target sequence on the target nucleic acid molecule may contain 1, 2, 3, 4, 5, 6, 7, 8, 9, or 10 mismatches. In some embodiments, the targeting sequence of the guide RNA and the target sequence on the target nucleic acid molecule may contain 1-6 mismatches. In some embodiments, the targeting sequence of the guide RNA and the target sequence on the target nucleic acid molecule may contain 5 or 6 mismatches.

The length of the targeting sequence may depend on the CRISPR/Cas9 system and components used. For example, different Cas9 proteins from different bacterial species have varying optimal targeting sequence lengths. Accordingly, the targeting sequence may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the targeting sequence may comprise 18-24 nucleotides in length. In some embodiments, the targeting sequence may comprise 19-21 nucleotides in length. In some embodiments, the targeting sequence may comprise 20 nucleotides in length.

The flagpole may comprise any sequence with sufficient complementarity with a tracr RNA to promote the formation of a functional CRISPR/Cas9 complex. In some embodiments, the flagpole may comprise all or a portion of the sequence (also called a “tag” or “handle”) of a naturally-occurring crRNA that is complementary to the tracr RNA in the same CRISPR/Cas9 system. In some embodiments, the flagpole may comprise all or a portion of a repeat sequence from a naturally-occurring CRISPR/Cas9 system. In some embodiments, the flagpole may comprise a truncated or modified tag or handle sequence. In some embodiments, the degree of complementarity between the tracr RNA and the portion of the flagpole that hybridizes with the tracr RNA along the length of the shorter of the two sequences may be about 40%, 50%, 60%, 70%, 80%, or higher, but lower than 100%. In some embodiments, the tracr RNA and the portion of the flagpole that hybridizes with the tracr RNA are not 100% complementary along the length of the shorter of the two sequences because of the presence of one or more bulge structures on the tracr and/or wobble base pairing between the tracr and the flagpole. The length of the flagpole may depend on the CRISPR/Cas9 system or the tracr RNA used. For example, the flagpole may comprise 10-50 nucleotides, or more than 50 nucleotides in length. In some embodiments, the flagpole may comprise 15-40 nucleotides in length. In other embodiments, the flagpole may comprise 20-30 nucleotides in length. In yet other embodiments, the flagpole may comprise 22 nucleotides in length. When a dual guide RNA is used, for example, the length of the flagpole may have no upper limit.

In some embodiments, the tracr RNA may comprise all or a portion of a wild-type tracr RNA sequence from a naturally-occurring CRISPR/Cas9 system. In some embodiments, the tracr RNA may comprise a truncated or modified variant of the wild-type tracr RNA. The length of the tracr RNA may depend on the CRISPR/Cas9 system used. In some embodiments, the tracr RNA may comprise 5, 6, 7, 8, 9, 10, 11, 12, 13, 14, 15, 16, 17, 18, 19, 20, 25, 30, 40, 50, 60, 70, 80, 90, 100, or more than 100 nucleotides in length. In certain embodiments, the tracr is at least 26 nucleotides in length. In additional embodiments, the tracr is at least 40 nucleotides in length. In some embodiments, the tracr RNA may comprise certain secondary structures, such as, e.g., one or more hairpins or stem-loop structures, or one or more bulge structures.

In some embodiments, the guide RNA may comprise two RNA molecules and is referred to herein as a “dual guide RNA” or “dgRNA”. In some embodiments, the dgRNA may comprise a first RNA molecule comprising a crRNA, and a second RNA molecule comprising a tracr RNA. The first and second RNA molecules may form a RNA duplex via the base pairing between the flagpole on the crRNA and the tracr RNA.

In additional embodiments, the guide RNA may comprise a single RNA molecule and is referred to herein as a “single guide RNA” or “sgRNA”. In some embodiments, the sgRNA may comprise a crRNA covalently linked to a tracr RNA. In some embodiments, the crRNA and the tracr RNA may be covalently linked via a linker. In some embodiments, the single-molecule guide RNA may comprise a stem-loop structure via the base pairing between the flagpole on the crRNA and the tracr RNA.

Certain embodiments of the invention also provide nucleic acids, e.g., vectors, encoding the guide RNA described herein. In some embodiments, the nucleic acid may be a DNA molecule. In other embodiments, the nucleic acid may be an RNA molecule. In some embodiments, the nucleic acid may comprise a nucleotide sequence encoding a crRNA. In some embodiments, the nucleotide sequence encoding the crRNA comprises a targeting sequence flanked by all or a portion of a repeat sequence from a naturally-occurring CRISPR/Cas system. In some embodiments, the nucleic acid may comprise a nucleotide sequence encoding a tracr RNA. In some embodiments, the crRNA and the tracr RNA may be encoded by two separate nucleic acids. In other embodiments, the crRNA and the tracr RNA may be encoded by a single nucleic acid. In some embodiments, the crRNA and the tracr RNA may be encoded by opposite strands of a single nucleic acid. In other embodiments, the crRNA and the tracr RNA may be encoded by the same strand of a single nucleic acid.

In certain embodiments, more than one guide RNA can be used with a CRISPR/Cas nuclease system. Each guide RNA may contain a different targeting sequence, such that the CRISPR/Cas system cleaves more than one target sequence. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA may be the same or different.

(b) Other Nuclease Systems

In additional embodiments, the nuclease in the nuclease systems described herein may be a nuclease other than a Cas protein. For example, the nuclease may be chosen from a meganuclease (e.g., homing endonucleases), ZFN, TALEN, and megaTAL.

Naturally-occurring meganucleases may recognize and cleave double-stranded DNA sequences of about 12 to 40 base pairs, and are commonly grouped into five families. In some embodiments, the meganuclease may be chosen from the LAGLIDADG family (SEQ ID NO: 1), the GIY-YIG family, the HNH family, the His-Cys box family, and the PD-(D/E)XK family. In some embodiments, the DNA binding domain of the meganuclease may be engineered to recognize and bind to a sequence other than its cognate target sequence. In some embodiments, the DNA binding domain of the meganuclease may be fused to a heterologous nuclease domain. In some embodiments, the meganuclease, such as a homing endonuclease, may be fused to TAL modules to create a hybrid protein, such as a “megaTAL” protein. The megaTAL protein may have improved DNA targeting specificity by recognizing the target sequences of both the DNA binding domain of the meganuclease and the TAL modules.

ZFNs are fusion proteins comprising a zinc-finger DNA binding domain (“zinc fingers” or “ZFs”) and a nuclease domain. Each naturally-occurring ZF may bind to three consecutive base pairs (a DNA triplet), and ZF repeats are combined to recognize a DNA target sequence and provide sufficient affinity. Thus, engineered ZF repeats may be combined to recognize longer DNA sequences, such as, e.g., 9-, 12-, 15-, or 18-bp, etc. In some embodiments, the ZFN may comprise ZFs fused to a nuclease domain from a restriction endonuclease. For example, the restriction endonuclease may be FokI. In some embodiments, the nuclease domain may comprise a dimerization domain, such as when the nuclease dimerizes to be active, and a pair of ZFNs comprising the ZF repeats and the nuclease domain may be designed for targeting a target sequence, which comprises two half target sequences recognized by each ZF repeats on opposite strands of the DNA molecule, with an interconnecting sequence in between (which is sometimes called a spacer in the literature). For example, the interconnecting sequence may be 5 to 7 bp in length. When both ZFNs of the pair bind, the nuclease domain may dimerize and introduce a DSB within the interconnecting sequence. In some embodiments, the dimerization domain of the nuclease domain may comprise a knob-into-hole motif to promote dimerization. For example, the ZFN may comprise a knob-into-hole motif in the dimerization domain of FokI.

The DNA binding domain of TALENs usually comprises a variable number of 34 or 35 amino acid repeats (“modules” or “TAL modules”), with each module binding to a single DNA base pair, A, T, G, or C. Adjacent residues at positions 12 and 13 (the “repeat-variable di-residue” or RVD) of each module specify the single DNA base pair that the module binds to. Though modules used to recognize G may also have affinity for A, TALENs benefit from a simple code of recognition—one module for each of the 4 bases—which greatly simplifies the customization of a DNA-binding domain recognizing a specific target sequence. In some embodiments, the TALEN may comprise a nuclease domain from a restriction endonuclease. For example, the restriction endonuclease may be FokI. In some embodiments, the nuclease domain may dimerize to be active, and a pair of TALENS may be designed for targeting a target sequence, which comprises two half target sequences recognized by each DNA binding domain on opposite strands of the DNA molecule, with an interconnecting sequence in between. For example, each half target sequence may be in the range of 10 to 20 bp, and the interconnecting sequence may be 12 to 19 bp in length. When both TALENs of the pair bind, the nuclease domain may dimerize and introduce a DSB within the interconnecting sequence. In some embodiments, the dimerization domain of the nuclease domain may comprise a knob-into-hole motif to promote dimerization. For example, the TALEN may comprise a knob-into-hole motif in the dimerization domain of FokI.

(c) Modified Nucleases

In certain embodiments, the nuclease may be optionally modified from its wild-type counterpart. In some embodiments, the nuclease may be fused with at least one heterologous protein domain. At least one protein domain may be located at the N-terminus, the C-terminus, or in an internal location of the nuclease. In some embodiments, two or more heterologous protein domains are at one or more locations on the nuclease.

In some embodiments, the protein domain may facilitate transport of the nuclease into the nucleus of a cell. For example, the protein domain may be a nuclear localization signal (NLS). In some embodiments, the nuclease may be fused with 1-10 NLS(s). In some embodiments, the nuclease may be fused with 1-5 NLS(s). In some embodiments, the nuclease may be fused with one NLS. In other embodiments, the nuclease may be fused with more than one NLS. In some embodiments, the nuclease may be fused with 2, 3, 4, or 5 NLSs. In some embodiments, the nuclease may be fused with 2 NLSs. In some embodiments, the nuclease may be fused with 3 NLSs. In some embodiments, the nuclease may be fused with no NLS. In some embodiments, the NLS may be a monopartite sequence, such as, e.g., the SV40 NLS, PKKKRKV (SEQ ID NO: 2) or PKKKRRV (SEQ ID NO: 3). In some embodiments, the NLS may be a bipartite sequence, such as, e.g., the NLS of nucleoplasmin, KRPAATKKAGQAKKKK (SEQ ID NO: 4). In some embodiments, the NLS may be genetically modified from its wild-type counterpart.

In some embodiments, the protein domain may be capable of modifying the intracellular half-life of the nuclease. In some embodiments, the half-life of the nuclease may be increased. In some embodiments, the half-life of the nuclease may be reduced. In some embodiments, the entity may be capable of increasing the stability of the nuclease. In some embodiments, the entity may be capable of reducing the stability of the nuclease. In some embodiments, the protein domain may act as a signal peptide for protein degradation. In some embodiments, the protein degradation may be mediated by proteolytic enzymes, such as, e.g., proteasomes, lysosomal proteases, or calpain proteases. In some embodiments, the protein domain may comprise a PEST sequence. In some embodiments, the nuclease may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub 1 in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBLS).

In some embodiments, the protein domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreenl), yellow fluorescent proteins (e.g., YFP, EYFP, Citrine, Venus, YPet, PhiYFP, ZsYellowl), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His (SEQ ID NO: 5), biotin carboxyl carrier protein (BCCP), and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the protein domain may target the nuclease to a specific organelle, cell type, tissue, or organ.

In further embodiments, the protein domain may be an effector domain. When the nuclease is directed to its target sequence, e.g., when a Cas9 protein is directed to a target sequence by a guide RNA, the effector domain may modify or affect the target sequence. In some embodiments, the effector domain may be chosen from a nucleic acid binding domain, a nuclease domain, an epigenetic modification domain, a transcriptional activation domain, or a transcriptional repressor domain.

Certain embodiments of the invention also provide nucleic acids encoding the nucleases (e.g., a Cas9 protein) described herein provided on a vector. In some embodiments, the nucleic acid may be a DNA molecule. In other embodiments, the nucleic acid may be an RNA molecule. In some embodiments, the nucleic acid encoding the nuclease may be an mRNA molecule. In certain embodiments, the nucleic acid is an mRNA encoding a Cas9 protein.

In some embodiments, the nucleic acid encoding the nuclease may be codon optimized for efficient expression in one or more eukaryotic cell types. In some embodiments, the nucleic acid encoding the nuclease may be codon optimized for efficient expression in one or more mammalian cells. In some embodiments, the nucleic acid encoding the nuclease may be codon optimized for efficient expression in human cells. Methods of codon optimization including codon usage tables and codon optimization algorithms are available in the art.

Target Sequences

The nuclease systems of the present disclosure may be directed to and cleave a target sequence on a target nucleic acid molecule. For example, the target sequence may be recognized and cleaved by the nuclease. In some embodiments, a Cas9 protein may be directed by a guide RNA to a target sequence of a target nucleic acid molecule, where the guide RNA hybridizes with and the Cas protein cleaves the target sequence. In some embodiments, the target sequence may be complementary to the targeting sequence of the guide RNA. In some embodiments, the degree of complementarity between a targeting sequence of a guide RNA and its corresponding target sequence may be about 50%, 55%, 60%, 65%, 70%, 75%, 80%, 85%, 90%, 95%, 97%, 98%, 99%, or 100%. In some embodiments, the target sequence and the targeting sequence of the guide RNA may be 100% complementary. In other embodiments, the target sequence and the targeting sequence of the guide RNA may contain at least one mismatch. For example, the target sequence and the targeting sequence of the guide RNA may contain 1, 2, 3, 4, 5, 6, 7, 8, 9, or 10 mismatches. In some embodiments, the target sequence and the targeting sequence of the guide RNA may contain 1-6 mismatches. In some embodiments, the target sequence and the targeting sequence of the guide RNA may contain 5 or 6 mismatches.

The length of the target sequence may depend on the nuclease system used. For example, the target sequence for a CRISPR/Cas system may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the target sequence may comprise 18-24 nucleotides in length. In some embodiments, the target sequence may comprise 19-21 nucleotides in length. In some embodiments, the target sequence may comprise 20 nucleotides in length. When nickases are used, the target sequence may comprise a pair of target sequences recognized by a pair of nickases on opposite strands of the DNA molecule.

In some embodiments, the target sequence for a meganuclease may comprise 12-40 or more nucleotides in length. When ZFNs are used, the target sequence may comprise two half target sequences recognized by a pair of ZFNs on opposite strands of the DNA molecule, with an interconnecting sequence in between. In some embodiments, each half target sequence for ZFNs may independently comprise 9, 12, 15, 18, or more nucleotides in length. In some embodiments, the interconnecting sequence for ZFNs may comprise 4-20 nucleotides in length. In some embodiments, the interconnecting sequence for ZFNs may comprise 5-7 nucleotides in length.

When TALENs are used, the target sequence may similarly comprise two half target sequences recognized by a pair of TALENs on opposite strands of the DNA molecule, with an interconnecting sequence in between. In some embodiments, each half target sequence for TALENs may independently comprise 10-20 or more nucleotides in length. In some embodiments, the interconnecting sequence for TALENs may comprise 4-20 nucleotides in length. In some embodiments, the interconnecting sequence for TALENs may comprise 12-19 nucleotides in length.

The target nucleic acid molecule may be any DNA or RNA molecule that is endogenous or exogenous to a cell. As used herein, the term “endogenous sequence” refers to a sequence that is native to the cell. The term “exogenous sequence” refers to a sequence that is not native to a cell, or a sequence whose native location in the genome of the cell is in a different location. In some embodiments, the target nucleic acid molecule may be a plasmid, a genomic DNA, or a chromosome from a cell or in the cell. In some embodiments, the target sequence of the target nucleic acid molecule may be a genomic sequence from a cell or in the cell. In some embodiments, the cell may be a prokaryotic cell. In other embodiments, the cell may be a eukaryotic cell. In some embodiments, the eukaryotic cell may be a mammalian cell. In some embodiments, the eukaryotic cell may be a rodent cell. In some embodiments, the eukaryotic cell may be a human cell. In further embodiments, the target sequence may be a viral sequence. In yet other embodiments, the target sequence may be a synthesized sequence. In some embodiments, the target sequence may be on a eukaryotic chromosome, such as a human chromosome.

In some embodiments, the target sequence may be located in a coding sequence of a gene, an intron sequence of a gene, a transcriptional control sequence of a gene, a translational control sequence of a gene, or a non-coding sequence between genes. In some embodiments, the gene may be a protein coding gene. In other embodiments, the gene may be a non-coding RNA gene. In some embodiments, the target sequence may comprise all or a portion of a disease-associated gene.

In some embodiments, the target sequence may be located in a non-genic functional site in the genome that controls aspects of chromatin organization, such as a scaffold site or locus control region. In some embodiments, the target sequence may be a genetic safe harbor site, i.e., a locus that facilitates safe genetic modification.

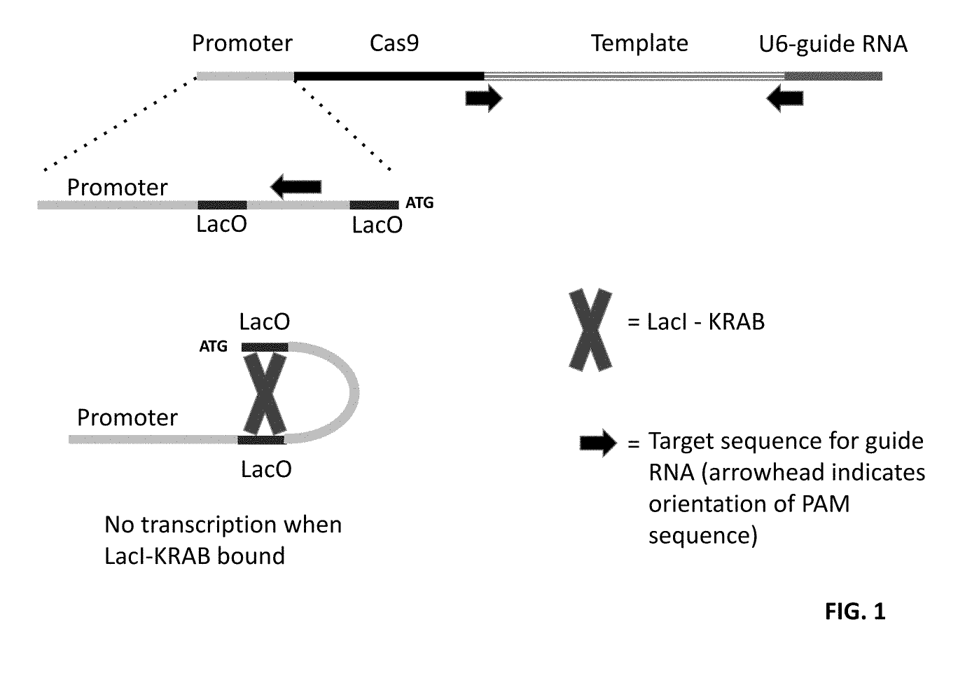

In some embodiments, the target sequence may be adjacent to a protospacer adjacent motif (PAM), a short sequence recognized by a CRISPR/Cas9 complex. In some embodiments, the PAM may be adjacent to or within 1, 2, 3, or 4, nucleotides of the 3′ end of the target sequence. The length and the sequence of the PAM may depend on the Cas9 protein used. For example, the PAM may be selected from a consensus or a particular PAM sequence for a specific Cas9 protein or Cas9 ortholog, including those disclosed in FIG. 1 of Ran et al., Nature, 520: 186-191 (2015), which is incorporated herein by reference. In some embodiments, the PAM may comprise 2, 3, 4, 5, 6, 7, 8, 9, or 10 nucleotides in length. Non-limiting exemplary PAM sequences include NGG, NGGNG, NG, NAAAAN, NNAAAAW, NNNNACA, GNNNCNNA, and NNNNGATT (wherein N is defined as any nucleotide, and W is defined as either A or T). In some embodiments, the PAM sequence may be NGG. In some embodiments, the PAM sequence may be NGGNG. In some embodiments, the PAM sequence may be NNAAAAW.

Templates

In some embodiments, at least one template may be provided as a substrate during the repair of the cleaved target nucleic acid molecule. In some embodiments, the template may be used in homologous recombination, such as, e.g., high-fidelity homologous recombination. In some embodiments, the homologous recombination may result in the integration of the template sequence into the target nucleic acid molecule. In some embodiments, a single template or multiple copies of the same template may be provided. In other embodiments, two or more templates may be provided such that homologous recombination may occur at two or more target sites. For example, different templates may be provided to repair a single gene in a cell, or two different genes in a cell. In some embodiments, the different templates may be provided in independent copy numbers.

In other embodiments, the template may be used in homology-directed repair, requiring DNA strand invasion at the site of the cleavage in the nucleic acid. In some embodiments, the homology-directed repair may result in the copying of the template sequence into the target nucleic acid molecule. In some embodiments, a single template or multiple copies of the same template may be provided. In other embodiments, two or more templates having different sequences may be inserted at two or more sites by homology-directed repair. For example, different templates may be provided to repair a single gene in a cell, or two different genes in a cell. In some embodiments, the different templates may be provided in independent copy numbers.

In yet other embodiments, the template may be incorporated into the cleaved nucleic acid as an insertion mediated by non-homologous end joining. In some embodiments, the template sequence has no similarity to the nucleic acid sequence near the cleavage site. In some embodiments, the template sequence (e.g., the coding sequence in the template) has no similarity to the nucleic acid sequence near the cleavage site. The template sequence may be flanked by target sequences that may have similar or identical sequence(s) to a target sequence near the cleavage site. In some embodiments, a single template or multiple copies of the same template may be provided. In other embodiments, two or more templates having different sequences may be inserted at two or more sites by non-homologous end joining. For example, different templates may be provided to insert a single template in a cell, or two different templates in a cell. In some embodiments, the different templates may be provided in independent copy numbers.

In some embodiments, the template sequence may correspond to an endogenous sequence of a target cell. In some embodiments, the endogenous sequence may be a genomic sequence of the cell. In some embodiments, the endogenous sequence may be a chromosomal or extrachromosomal sequence. In some embodiments, the endogenous sequence may be a plasmid sequence of the cell. In some embodiments, the template sequence may be substantially identical to a portion of the endogenous sequence in a cell at or near the cleavage site, but comprise at least one nucleotide change. In some embodiments, the repair of the cleaved target nucleic acid molecule with the template may result in a mutation comprising an insertion, deletion, or substitution of one or more nucleotides of the target nucleic acid molecule. In some embodiments, the mutation may result in one or more amino acid changes in a protein expressed from a gene comprising the target sequence. In some embodiments, the mutation may result in one or more nucleotide changes in an RNA expressed from the target gene. In some embodiments, the mutation may alter the expression level of the target gene. In some embodiments, the mutation may result in increased or decreased expression of the target gene. In some embodiments, the mutation may result in gene knockdown. In some embodiments, the mutation may result in gene knockout. In some embodiments, the repair of the cleaved target nucleic acid molecule with the template may result in replacement of an exon sequence, an intron sequence, a transcriptional control sequence, a translational control sequence, or a non-coding sequence of the target gene.

In other embodiments, the template sequence may comprise an exogenous sequence. In some embodiments, the exogenous sequence may comprise a protein or RNA coding sequence operably linked to an exogenous promoter sequence such that, upon integration of the exogenous sequence into the target nucleic acid molecule, the cell is capable of expressing the protein or RNA encoded by the integrated sequence. In other embodiments, upon integration of the exogenous sequence into the target nucleic acid molecule, the expression of the integrated sequence may be regulated by an endogenous promoter sequence. In some embodiments, the exogenous sequence may be a chromosomal or extrachromosomal sequence. In some embodiments, the exogenous sequence may provide a cDNA sequence encoding a protein or a portion of the protein. In yet other embodiments, the exogenous sequence may comprise an exon sequence, an intron sequence, a transcriptional control sequence, a translational control sequence, or a non-coding sequence. In some embodiments, the integration of the exogenous sequence may result in gene knock-in.

The template may be of any suitable length. In some embodiments, the template may comprise 10, 15, 20, 25, 50, 75, 100, 150, 200, 500, 1000, 1500, 2000, 2500, 3000, 3500, 4000, 4500, 5000, 5500, 6000, or more nucleotides in length. In some embodiments, the template may comprise a nucleotide sequence that is complementary to a portion of the target nucleic acid molecule comprising the target sequence (i.e., a “homology arm”). In some embodiments, a homology arm may comprise 10, 15, 20, 25, 50, 75, 100, 150, 200, 500, 1000, 1500, 2000, 2500, 3000 or more nucleotides in length. In some embodiments, the template may comprise a homology arm that is complementary to the sequence located upstream or downstream of the cleavage site on the target nucleic acid molecule. In some embodiments, the template may comprise a first nucleotide sequence and a second homology arm that are complementary to the sequences located upstream and downstream of the cleavage site, respectively. Where a template contains two homology arms, each arm can be the same length or different lengths, and the sequence between the homology arms can be substantially similar or identical to the target sequence between the homology arms, or be entirely unrelated. In some embodiments, the degree of complementarity between the first nucleotide sequence on the template and the sequence upstream of the cleavage site, and between the second nucleotide sequence on the template and the sequence downstream of the cleavage site, may permit homologous recombination, such as, e.g., high-fidelity homologous recombination, between the template and the target nucleic acid molecule. In some embodiments, the degree of complementarity may be about 50%, 55%, 60%, 65%, 70%, 75%, 80%, 85%, 90%, 95%, 97%, 98%, 99%, or 100%. In some embodiments, the degree of complementarity may be about 95%, 97%, 98%, 99%, or 100%. In some embodiments, the degree of complementarity may be about 98%, 99%, or 100%. In some embodiments, the degree of complementarity may be 100%. In some embodiments, for example those described herein where a template is incorporated into the cleaved nucleic acid as an insertion mediated by non-homologous end joining, the template has no homology arms. In some embodiments, a template having no homology arms comprises target sequences flanking one or both ends of the template sequence, e.g., as described herein. In some embodiments, a template having no homology arms comprises target sequences flanking both ends of the template sequence. In some embodiments, a target sequence flanking the end of the template sequence is about 10-50 nucleotides. In some embodiments, a target sequence flanking the end of the template sequence is about 10-20 nucleotides, about 15-20 nucleotides, about 20-25 nucleotides, or about 20-30 nucleotides. In some embodiments, a target sequence flanking the end of the template sequence is about 17-23 nucleotides. In some embodiments, a target sequence flanking the end of the template sequence is about 20 nucleotides.

In some embodiments, a nucleic acid molecule is expressed from the template if homologous recombination occurs between the template and the genomic sequence. In some embodiments, for example, the template does not have a promoter for expressing the nucleic acid molecule and/or the ATG transcriptional start site is removed from the coding sequence.

Vectors

In some embodiments, the nuclease system and the template may be provided on one or more vectors. In some embodiments, the vector may be a DNA vector. In other embodiments, the vector may be an RNA vector. In some embodiments, the RNA vector may be an mRNA, e.g. an mRNA that encodes a nuclease such as Cas9. See, e.g., Tolmachov et al., Gene Technology, 4(1) (2015). In some embodiments, the vector may be circular. In other embodiments, the vector may be linear. Non-limiting exemplary vectors include plasmids, phagemids, cosmids, artificial chromosomes, minichromosomes, transposons, viral vectors, and expression vectors. In some embodiments, the nuclease is provided by an RNA vector, e.g., as mRNA, and the template is provided by a viral vector.

In some embodiments, the vector may be a viral vector. In some embodiments, the viral vector may be genetically modified from its wild-type counterpart. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some embodiments, a portion of the viral genome may be deleted such that the virus is capable of packaging exogenous sequences having a larger size. In some embodiments, the viral vector may have an enhanced transduction efficiency. In some embodiments, the immune response induced by the virus in a host may be reduced. In some embodiments, viral genes (such as, e.g., integrase) that promote integration of the viral sequence into a host genome may be mutated such that the virus becomes non-integrating. In some embodiments, the viral vector may be replication defective. In some embodiments, the viral vector may comprise exogenous transcriptional or translational control sequences to drive expression of coding sequences on the vector. In some embodiments, the virus may be helper-dependent. For example, the virus may need one or more helper virus to supply viral components (such as, e.g., viral proteins) required to amplify and package the vectors into viral particles. In such a case, one or more helper components, including one or more vectors encoding the viral components, may be introduced into a host cell along with the vector system described herein. In other embodiments, the virus may be helper-free. For example, the virus may be capable of amplifying and packaging the vectors without any helper virus. In some embodiments, the vector system described herein may also encode the viral components required for virus amplification and packaging.

Non-limiting exemplary viral vectors include adeno-associated virus (AAV) vector, lentivirus vectors, adenovirus vectors, herpes simplex virus (HSV-1) vectors, bacteriophage T4, baculovirus vectors, and retrovirus vectors. In some embodiments, the viral vector may be an AAV vector. In other embodiments, the viral vector may a lentivirus vector. In some embodiments, the lentivirus may be non-integrating. In some embodiments, the viral vector may be an adenovirus vector. In some embodiments, the adenovirus may be a high-cloning capacity or “gutless” adenovirus, where all coding viral regions apart from the 5′ and 3′ inverted terminal repeats (ITRs) and the packaging signal (Ψ) are deleted from the virus to increase its packaging capacity. In yet other embodiments, the viral vector may be an HSV-1 vector. In some embodiments, the HSV-1-based vector is helper dependent, and in other embodiments it is helper independent. For example, an amplicon vector that retains only the packaging sequence requires a helper virus with structural components for packaging, while a 30 kb-deleted HSV-1 vector that removes non-essential viral functions does not require helper virus. In additional embodiments, the viral vector may be bacteriophage T4. In some embodiments, the bacteriophage T4 may be able to package any linear or circular DNA or RNA molecules when the head of the virus is emptied. In further embodiments, the viral vector may be a baculovirus vector. In yet further embodiments, the viral vector may be a retrovirus vector. In embodiments using AAV or lentiviral vectors, which have smaller cloning capacity, it may be necessary to use more than one vector to deliver all the components of a vector system as disclosed herein. For example, one AAV vector may contain sequences encoding a Cas9 protein, while a second AAV vector may contain one or more guide sequences and one or more copies of template.

In certain embodiments, a viral vector may be modified to target a particular tissue or cell type. For example, viral surface proteins may be altered to decrease or eliminate viral protein binding to its natural cell surface receptor(s). The surface proteins may also be engineered to interact with a receptor specific to a desired cell type. Viral vectors may have altered host tropism, including limited or redirected tropism. Certain engineered viral vectors are described, for example, in WO2011130749 [HSV], WO2015009952 [HSV], U.S. Pat. No. 5,817,491 [retrovirus], WO2014135998 [T4], and WO2011125054 [T4], each of which is incorporated herein by reference for its engineered viral vectors. In some embodiments, the viral vector may be engineered to express or display a first binding moiety. The first binding moiety may be fused to a viral surface protein or glycoprotein, conjugated to a virus, chemically crosslinked to a virion, bound to a virus envelope, or joined to a viral vector by any other suitable method. The first binding moiety is capable of binding to a second binding moiety, which may be used to direct the virus to a desired cell type. In some embodiments, the first binding moiety is avidin, streptavidin, neutravidin, captavidin, or another biotin-binding moiety, and the second binding moiety is biotin or an analog thereof. A biotinylated targeting agent may then be bound to the avidin on the viral vector and used to direct the virus to a desired cell type. For example, a T4 vector may be engineered to display a biotin-binding moiety on one or more of its surface proteins. The cell-specificity of such a T4 vector may then be altered by binding a biotinylated antibody or ligand directed to a cell of choice. In alternate embodiments, the first and second binding moieties are hapten and an anti-hapten binding protein; digoxigenin and an anti-digoxigenin binding protein; fluorescein and an anti-fluorescein binding protein; or any other suitable first and second binding moieties that are binding partners.

In some embodiments, the vector may be capable of driving expression of one or more coding sequences in a cell. In some embodiments, the cell may be a prokaryotic cell, such as, e.g., a bacterial cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild-type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the vector may comprise a nucleotide sequence encoding the nuclease described herein. In some embodiments, the vector system may comprise one copy of the nucleotide sequence encoding the nuclease. In other embodiments, the vector system may comprise more than one copy of the nucleotide sequence encoding the nuclease. In some embodiments, the nucleotide sequence encoding the nuclease may be operably linked to at least one transcriptional or translational control sequence. In some embodiments, the nucleotide sequence encoding the nuclease may be operably linked to at least one promoter. In some embodiments, the nucleotide sequence encoding the nuclease may be operably linked to at least one transcriptional or translational control sequence.

In some embodiments, the promoter may be constitutive, inducible, or tissue-specific. In some embodiments, the promoter may be a constitutive promoter. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1α) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EF1α promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech). In some embodiments, the promoter may be a tissue-specific promoter. In some embodiments, the tissue-specific promoter is exclusively or predominantly expressed in liver tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphsl promoter, OG-2 promoter, SP-B promoter, SYN1 promoter, and WASP promoter.

In some embodiments, the nuclease encoded by the vector may be a Cas protein, such as a Cas9 protein or Cpf1 protein. The vector system may further comprise a vector comprising a nucleotide sequence encoding the guide RNA described herein. In some embodiments, the vector system may comprise one copy of the guide RNA. In other embodiments, the vector system may comprise more than one copy of the guide RNA. In embodiments with more than one guide RNA, the guide RNAs may be non-identical such that they target different target sequences, or have other different properties, such as activity or stability within the Cas9 RNP complex. In some embodiments, the nucleotide sequence encoding the guide RNA may be operably linked to at least one transcriptional or translational control sequence. In some embodiments, the nucleotide sequence encoding the guide RNA may be operably linked to at least one promoter. In some embodiments, the promoter may be recognized by RNA polymerase III (Pol III). Non-limiting examples of Pol III promoters include U6, H1 and tRNA promoters. In some embodiments, the nucleotide sequence encoding the guide RNA may be operably linked to a mouse or human U6 promoter. In other embodiments, the nucleotide sequence encoding the guide RNA may be operably linked to a mouse or human H1 promoter. In some embodiments, the nucleotide sequence encoding the guide RNA may be operably linked to a mouse or human tRNA promoter. In embodiments with more than one guide RNA, the promoters used to drive expression may be the same or different. In some embodiments, the nucleotide encoding the crRNA of the guide RNA and the nucleotide encoding the tracr RNA of the guide RNA may be provided on the same vector. In some embodiments, the nucleotide encoding the crRNA and the nucleotide encoding the tracr RNA may be driven by the same promoter. In some embodiments, the crRNA and tracr RNA may be transcribed into a single transcript. For example, the crRNA and tracr RNA may be processed from the single transcript to form a double-molecule guide RNA. Alternatively, the crRNA and tracr RNA may be transcribed into a single-molecule guide RNA. In other embodiments, the crRNA and the tracr RNA may be driven by their corresponding promoters on the same vector. In yet other embodiments, the crRNA and the tracr RNA may be encoded by different vectors.

In some embodiments, the nucleotide sequence encoding the guide RNA may be located on the same vector comprising the nucleotide sequence encoding a Cas9 protein. In some embodiments, expression of the guide RNA and of the Cas9 protein may be driven by their corresponding promoters. In some embodiments, expression of the guide RNA may be driven by the same promoter that drives expression of the Cas9 protein. In some embodiments, the guide RNA and the Cas9 protein transcript may be contained within a single transcript. For example, the guide RNA may be within an untranslated region (UTR) of the Cas9 protein transcript. In some embodiments, the guide RNA may be within the 5′ UTR of the Cas9 protein transcript. In other embodiments, the guide RNA may be within the 3′ UTR of the Cas9 protein transcript. In some embodiments, the intracellular half-life of the Cas9 protein transcript may be reduced by containing the guide RNA within its 3′ UTR and thereby shortening the length of its 3′ UTR. In additional embodiments, the guide RNA may be within an intron of the Cas9 protein transcript. In some embodiments, suitable splice sites may be added at the intron within which the guide RNA is located such that the guide RNA is properly spliced out of the transcript. In some embodiments, expression of the Cas9 protein and the guide RNA in close proximity on the same vector may facilitate more efficient formation of the CRISPR complex.

In some embodiments, the vector system may further comprise a vector comprising the template described herein. In some embodiments, the vector system may comprise one copy of the template. In other embodiments, the vector system may comprise more than one copy of the template. In some embodiments, the vector system may comprise 2, 3, 4, 5, 6, 7, 8, 9, 10, or more copies of the template. In some embodiments, the vector system may comprise 4, 5, 6, 7, 8, or more copies of the template. In some embodiments, the vector system may comprise 5, 6, 7, or more copies of the template. In some embodiments, the vector system may comprise 6 copies of the template. The multiple copies of the template may be located on the same or different vectors. The multiple copies of the template may also be adjacent to one another, or separated by other nucleotide sequences or vector elements. In other embodiments, two or more templates may be provided such that homologous recombination may occur at two or more target sites. For example, different templates may be provided to repair a single gene in a cell, or two different genes in a cell. In some embodiments, the different templates may be provided in independent copy numbers.

A vector system may comprise 1-3 vectors. In some embodiments, the vector system may comprise one single vector. In other embodiments, the vector system may comprise two vectors. In additional embodiments, the vector system may comprise three vectors. When different guide RNAs or templates are used for multiplexing, or when multiple copies of the guide RNA or the template are used, the vector system may comprise more than three vectors.

In some embodiments, the nucleotide sequence encoding the nuclease and the template may be located on the same or separate vectors. In some embodiments, the nucleotide sequence encoding the nuclease and the template may be located on the same vector. In some embodiments, the nucleotide sequence encoding the nuclease and the template may be located on separate vectors. The sequences may be oriented in the same or different directions and in any order on the vector.

In some embodiments, the nucleotide sequence encoding a Cas9 protein, a nucleotide sequence encoding the guide RNA, and a template may be located on the same or separate vectors. In some embodiments, all of the sequences may be located on the same vector. In some embodiments, two or more sequences may be located on the same vector. The sequences may be oriented in the same or different directions and in any order on the vector. In some embodiments, the nucleotide sequence encoding the Cas9 protein and the nucleotide sequence encoding the guide RNA may be located on the same vector. In some embodiments, the nucleotide sequence encoding the Cas9 protein and the template may be located on the same vector. In some embodiments, the nucleotide sequence encoding the guide RNA and the template may be located on the same vector. In a particular embodiment, the vector system may comprise a first vector comprising the nucleotide sequence encoding the Cas9 protein, and a second vector comprising the nucleotide sequence encoding the guide RNA and the template or multiple copies of the template.

In some embodiments, the template may be released from the vector on which it is located by the nuclease system encoded by the vector system. For example, the template may be released from the vector by a Cas9 protein and a guide RNA encoded by the vector system. In other embodiments, the template may be released from the vector by a Cas9 protein and a guide RNA that are not encoded in a viral vector. In some embodiments, the template may be released from the vector by a Cas9 protein provided from an mRNA. The template may comprise at least one target sequence that is recognized by the guide RNA. In some embodiments, the template may be flanked by a target sequence at the 5′ and 3′ ends of the template. Upon expression of Cas9 protein and guide RNA, the guide RNA may hybridize with and the Cas9 protein may cleave the target sequence at both ends of the template such that the template is released from the vector. In additional embodiments, the template may be released from the vector by a nuclease encoded by the vector system by having a target sequence recognized by the nuclease at the 5′ and 3′ ends of the template. The target sequences at either end of the template may be oriented such that the PAM sequence is closer to the template. In such an orientation, fewer non-template nucleic acids remain on the ends of the template after release from the vector. In some embodiments, the target sequences flanking the template may be the same. In some embodiments, the target sequences flanking the template may be the same as the target sequence found at the cleavage site in which the template is incorporated, e.g., by HR, HDR, or non-homologous end joining. In other embodiments, the target sequences flanking the template may be different. For example, the target sequence at the 5′ end of the template may be recognized by one guide RNA or nuclease, and the target sequence at the 3′ end of the template may be recognized by another guide RNA or nuclease.

In some embodiments, the vector encoding the nuclease system may comprise at least one target sequence within the vector, to create a self-destroying (or “self-cleaving” or “self-inactivating”) vector system to control the amount of the nuclease system to be expressed. In some embodiments, the self-destroying vector system results in a reduction in the amount of nuclease activity. In further embodiments, the self-destroying vector system results in a reduction in the amount of vector nucleic acid. In embodiments in which the system comprises Cas9, it also comprises guide RNA(s) that recognize the target sequence. In this way, the residence time and/or the level of activity of the nuclease system may be temporally controlled to avoid adverse effects associated with overexpression of the nuclease system. Such adverse effects may include, e.g., an off-target effect by the nuclease. In some embodiments, one or more target sequences may be located at any place on the vector such that, upon expression of the nuclease, the nuclease recognizes and cleaves the target sequence in the vector that contains the nuclease-encoding sequence. The one or more target sequences of the self-destroying vector may be the same. Optionally, the self-destroying vector may comprise multiple target sequences. In some embodiments, the cleavage at a target sequence may reduce the expression of at least one component of the nuclease system, such as, for example, Cas9. In some embodiments, the cleavage may reduce the expression of the nuclease transcript. For example, a target sequence may be located within the nucleotide sequence encoding the nuclease such that the cleavage results in the disruption of the coding region. In other embodiments, a target sequence may be located within a non-coding region on the vector encoding the nuclease. In some embodiments, a target sequence may be located within the promoter that drives the expression of the nuclease such that the cleavage results in the disruption of the promoter sequence. For example, the vector may contain a target sequence (and its corresponding guide RNA) that targets a Cas9 sequence. In certain embodiments, a target sequence may be located between the promoter and the nucleotide sequence encoding the nuclease such that the cleavage results in the separation of the coding sequence from its promoter. In certain embodiments, a target sequence outside the nuclease coding sequence and a target sequence within the nuclease coding sequence are included.

In some embodiments, the vector comprises multiple cleavage sites in addition to the target sequences described for releasing the template and for self-cleaving. In some instances, the vector may be repaired instead of degraded if cleavage is insufficient or incomplete. In some embodiments, vector degradation is at least 70%, 80%, 90%, 95%, 96%, 97%, 98%, 99%, or 99.5%. Thus, in some embodiments, the vector comprises one, two, three, four, five, six, seven, eight, nine, ten, or more additional cleavage sites.

In some embodiments, the vector encoding a Cas9 protein may comprise at least one target sequence that is recognized by a guide RNA. In some embodiments, the target sequence may be located at any place on the vector such that, upon expression of the Cas9 protein and the guide RNA, the guide RNA hybridizes with and the Cas9 protein cleaves the target sequence in the vector encoding the Cas9 protein. In some embodiments, the cleavage at the target sequence may reduce the expression of the Cas9 protein transcript. For example, the target sequence may be located within the nucleotide sequence encoding the Cas9 protein such that the cleavage results in the disruption of the coding region. In other embodiments, the target sequence may be located within a non-coding region on the vector encoding the Cas9 protein. In some embodiments, the target sequence may be located within the promoter that drives the expression of the Cas9 protein such that the cleavage results in the disruption of the promoter sequence. In some embodiments, the target sequence may be located within the nucleotide sequence encoding the Cas9 protein such that the cleavage results in the disruption of the coding sequence. In other embodiments, the target sequence may be located between the promoter and the nucleotide sequence encoding the Cas9 protein such that the cleavage results in the separation of the coding sequence from its promoter.

In additional embodiments, the vector encoding the guide RNA may comprise at least one target sequence that is recognized by a guide RNA of the nuclease system. In some embodiments, the target sequence may be located at any place on the vector such that, upon expression of a Cas9 protein and the guide RNA, the guide RNA hybridizes with and the Cas9 protein cleaves the target sequence in the vector encoding the guide RNA. In some embodiments, the cleavage at the target sequence may reduce the expression of the guide RNA. In other embodiments, the target sequence may be located within a non-coding region on the vector encoding the guide RNA. In some embodiments, the target sequence may be located within the promoter that drives the expression of the guide RNA such that the cleavage results in the disruption of the promoter sequence. In other embodiments, the target sequence may be located between the promoter and the nucleotide sequence encoding the guide RNA such that the cleavage results in the separation of the coding sequence from its promoter.

The target sequences for release of the template, for vector self-destruction, and for targeting by the nuclease system in a cell may be the same or different. For example, the target sequence at the 3′ end of the template may be present within the promoter driving the expression of the nuclease (e.g., the Cas9 protein) or the guide RNA such that the release of the template simultaneously results in the disruption of the expression of either the nuclease (e.g., the Cas9 protein) or the guide RNA. In some embodiments, both target sequences flanking the template, the target sequences for disrupting the expression of the nuclease (e.g., the Cas9 protein), and the target sequence in the target nucleic acid molecule in a cell may be the same sequence that is recognized by a single guide RNA or nuclease. Thus, in some embodiments, the vector system may comprise only one type of target sequence, and the nuclease system may comprise only one guide RNA. In other embodiments, these target sequences may comprise different sequences that are recognized by different guide RNAs.

Accordingly, in some embodiments of the present disclosure, expression of the nuclease system may result in fragmentation of the encoding vectors, a process we name “crisprthripsis”. When the nuclease system and the template are encoded by a single viral vector, the vector fragmentation may also affect virus production when the vectors are amplified in host cells for growing the virus, for example due to some amount of nuclease being expressed during viral production. Therefore, the vector system may further comprise a mechanism to shut down expression of at least one component of the nuclease system before the vector system is delivered to a target cell. For example, the mechanism may be used to shut down expression of the nuclease (e.g., the Cas9 protein) and/or the guide RNA. In some embodiments, the expression of the vector system may be shut down during virus production.

For example, the vector system may comprise a lac operator (lacO)/lac repressor (LacI) system to prevent transcription. In some embodiments, the vector encoding the nuclease (e.g., the Cas9 protein) may comprise at least two lacO sequences within the promoter which drives the expression of the nuclease. In other embodiments, the vector may comprise at least two lacO sequences between the promoter and the nucleotide sequence encoding the nuclease. In some embodiments, the vector encoding the guide RNA may comprise at least two lacO sequences within the promoter that drives the expression of the guide RNA. In other embodiments, the vector may comprise at least two lacO sequences between the promoter and the nucleotide sequence encoding the guide RNA. In some embodiments, the at least two lacO sequences may flank a target sequence for self-destroying the vector. In some embodiments, the vector may comprise at least two sets of lacO repeats, wherein each set of the lacO repeats may comprise two lacO sequences. In some embodiments, two lacO sequences or the two sets of lacO repeats may be 30, 40 50, 60, 70, or 80 nucleotides apart. In additional embodiments, two lacO sequences are 55, 56, 57, 58, 59, or 60 nucleotides apart, as measured from the center of one lacO sequence to the center of a second lacO sequence. In some embodiments, the LacI may be encoded by and expressed from the same vector on which the lacO is located. In other embodiments, the LacI may be provided by a separate vector. In yet other embodiments, the LacI may be expressed in a cell where the vector system is amplified for production before delivery into a target cell. In those embodiments using viral vectors, the LacI may be expressed in the production host cell. In some embodiments, the LacI may be constitutively expressed in the production host cell. In other embodiments, the LacI may be transiently expressed in the production host cell. During amplification of the vector system or during virus production, the lacO and LacI may form a complex on the vector DNA that encodes the nuclease, or the guide RNA, or both. Without being bound by any theory, the lacO/LacI complex may interfere with transcription initiation by steric hindrance at the promoter. In some embodiments, the LacI may be fused to a transcription repressor domain to further enhance transcriptional inhibition. For example, the LacI may be fused to a Krüppel associated box (KRAB) domain.

Thus, certain embodiments of the invention include methods for producing a virus comprising the vector system described herein. In some embodiments, the method may comprise providing a cell expressing a LacI protein; introducing the vector system into the cell; introducing into the cell one or more viral components for producing the virus; growing the cell, and isolating the virus comprising the vector system from the cell. In other embodiments, the method may comprise introducing into a cell a vector comprising a nucleic acid sequence encoding a LacI protein, the vector system, and one or more viral components for producing the virus; growing the cell; and isolating the virus comprising the vector system from the cell. In some embodiments, the LacI protein may be fused to a KRAB domain. In some embodiments, the one or more viral components may be encoded by the vector system. In other embodiments, the one or more viral components may be introduced via a separate vector other than the vector system. In some embodiments, the method may further comprise adding an agent to remove the LacI bound to the lacO during or after isolation of the vector system from the cell culture. In some embodiments, the agent may be Isopropyl β-D-1-thiogalactopyranoside (IPTG). In some embodiment, the agent may be lactose.

In some embodiments, the vector system may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the vector system may comprise tissue-specific promoters to start expression only after it is delivered into a specific tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphsl promoter, OG-2 promoter, SP-B promoter, SYN1 promoter, and WASP promoter.

Temporal Regulation of System Activity

In some embodiments of the present disclosure, the activity of the nuclease system may be temporally regulated by adjusting the residence time, the amount, and/or the activity of the expressed components of the nuclease system. For example, as described herein, the nuclease may be fused with a protein domain that is capable of modifying the intracellular half-life of the nuclease. In certain embodiments involving two or more vectors (e.g., a vector system in which the components described herein are encoded on two or more separate vectors), the activity of the nuclease system may be temporally regulated by controlling the timing in which the vectors are delivered. For example, in some embodiments a vector encoding the nuclease system may deliver the nuclease prior to the vector encoding the template. In other embodiments, the vector encoding the template may deliver the template prior to the vector encoding the nuclease system. In some embodiments, the vectors encoding the nuclease system and template are delivered simultaneously. In certain embodiments, the simultaneously delivered vectors temporally deliver, e.g., the nuclease, template, and/or guide RNA components. In further embodiments, the RNA (such as, e.g., the nuclease transcript) transcribed from the coding sequence on the vectors may further comprise at least one element that is capable of modifying the intracellular half-life of the RNA and/or modulating translational control. In some embodiments, the half-life of the RNA may be increased. In some embodiments, the half-life of the RNA may be decreased. In some embodiments, the element may be capable of increasing the stability of the RNA. In some embodiments, the element may be capable of decreasing the stability of the RNA. In some embodiments, the element may be within the 3′ UTR of the RNA. In some embodiments, the element may include a polyadenylation signal (PA). In some embodiments, the element may include a cap, e.g., an upstream mRNA end. In some embodiments, the PA may be added to the 3′ UTR of the RNA. In some embodiments, the RNA may comprise no PA such that it is subject to quicker degradation in the cell after transcription. In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus (WHP) Posttranscriptional Regulatory Element (WPRE), which creates a tertiary structure to enhance expression from the transcript. In further embodiments, the element is a modified and/or truncated WPRE sequence that is capable of enhancing expression from the transcript, as described, for example in Zufferey et al., J Virol, 73(4): 2886-92 (1999) and Flajolet et al., J Virol, 72(7): 6175-80 (1998). In some embodiments, the WPRE or equivalent may be added to the 3′ UTR of the RNA. In some embodiments, the element may be selected from other RNA sequence motifs that are enriched in either fast- or slow-decaying transcripts.

In some embodiments, the vector encoding the nuclease or the guide RNA may be self-destroyed via cleavage of a target sequence present on the vector by the nuclease system. The cleavage may prevent continued transcription of a nuclease or a guide RNA from the vector. Although transcription may occur on the linearized vector for some amount of time, the expressed transcripts or proteins subject to intracellular degradation will have less time to produce off-target effects without continued supply from expression of the encoding vectors.

In some embodiments, the target sequences for template release, for vector self-destruction, and for targeting by the nuclease system in a cell may be the same that is recognized by a single guide RNA or a single nuclease. Thus, these three events may occur contemporaneously such that the timing of template release, disruption of the expression of the vector system, and cleavage of the target nucleic acid molecule are coordinated. In some embodiments, the guide RNA used to release the template and cleave the expression vector can be the same guide RNA that targets the desired genomic site. In additional embodiments, more than one guide RNA is used to achieve the various cleavage events.

In other embodiments, the guide RNA and the target sequence on the target nucleic acid molecule in a cell may contain at least one mismatch such that the cleavage by the Cas9 protein may be less efficient. In this way, the timing and persistence of Cas9 production can be controlled. In yet other embodiments, the nuclease system may use different guide RNAs to mediate DNA cleavage by the Cas protein. With different binding efficiencies between the Cas protein and the different guide RNAs, the timing of cleavage at the corresponding target sequences may be further regulated.

Combinations of some or all of the above mechanisms are also encompassed. For example, a combination may facilitate temporal control of the activity of the nuclease system to improve gene editing results, by reducing adverse effects (e.g., off-target effects) associated with overexpression of the nuclease or prolonged duration of the enzyme activity. The activity of the nuclease system may be monitored in real time by determining the amount or activity of the nuclease, the RNA transcript, or the vector. In some embodiments, the methods are quantitative. The cleavage or HR events on the target nucleic acid molecule may be also monitored over time by, e.g., real-time PCR.

Methods of Treatment

Embodiments of the invention encompass methods for editing a nucleic acid molecule in a cell. In some embodiments, the method may comprise introducing the vector system described herein into a cell. In some embodiments, the introduction of the vector system into the cell may result in a stable cell line having the edited nucleic acid molecule while the vectors are lost, e.g., targeted for self-destruction. In some embodiments, the cell is a eukaryotic cell. Non-limiting examples of eukaryotic cells include yeast cells, plant cells, insect cells, cells from an invertebrate animal, cells from a vertebrate animal, mammalian cells, rodent cells, mouse cells, rat cells, and human cells. In some embodiments, the eukaryotic cell may be a mammalian cell. In some embodiments, the eukaryotic cell may be a rodent cell. In some embodiments, the eukaryotic cell may be a human cell. Similarly, the target sequence may be from any such cells or in any such cells.

The vector system may be introduced into the cell via any methods known in the art, such as, e.g., viral or bacteriophage infection, transfection, conjugation, protoplast fusion, lipofection, electroporation, calcium phosphate precipitation, polyethyleneimine (PEI)-mediated transfection, DEAE-dextran-mediated transfection, liposome-mediated transfection, particle gun technology, calcium phosphate precipitation, shear-driven cell permeation, fusion to a cell-penetrating peptide followed by cell contact, microinjection, and nanoparticle-mediated delivery. In some embodiments, the vector system may be introduced into the cell via viral infection. In some embodiments, the vector system may be introduced into the cell via bacteriophage infection.

Embodiments of the invention also encompass treating a patient with the vector system described herein. In some embodiments, the method may comprise administering the vector system described herein to the patient. The method may be used as a single therapy or in combination with other therapies available in the art. In some embodiments, the patient may have a mutation (such as, e.g., insertion, deletion, substitution, chromosome translocation) in a disease-associated gene. In some embodiments, administration of the vector system may result in a mutation comprising an insertion, deletion, or substitution of one or more nucleotides of the disease-associated gene in the patient. Certain embodiments may include methods of repairing the patient's mutation in the disease-associated gene. In some embodiments, the mutation may result in one or more amino acid changes in a protein expressed from the disease-associated gene. In some embodiments, the mutation may result in one or more nucleotide changes in an RNA expressed from the disease-associated gene. In some embodiments, the mutation may alter the expression level of the disease-associated gene. In some embodiments, the mutation may result in increased or decreased expression of the gene. In some embodiments, the mutation may result in gene knockdown in the patient. In some embodiments, the administration of the vector system may result in the correction of the patient's mutation in the disease-associated gene. In some embodiments, the administration of the vector system may result in gene knockout in the patient. In some embodiments, the administration of the vector system may result in replacement of an exon sequence, an intron sequence, a transcriptional control sequence, a translational control sequence, or a non-coding sequence of the disease-associated gene.

In some embodiments, the administration of the vector system may result in integration of an exogenous sequence of the template into the patient's genomic DNA. In some embodiments, the exogenous sequence may comprise a protein or RNA coding sequence operably linked to an exogenous promoter sequence such that, upon integration of the exogenous sequence into the patient's genomic DNA, the patient is capable of expressing the protein or RNA encoded by the integrated sequence. The exogenous sequence may provide a supplemental or replacement protein coding or non-coding sequence. For example, the administration of the vector system may result in the replacement of the mutant portion of the disease-associated gene in the patient. In some embodiments, the mutant portion may include an exon of the disease-associated gene. In other embodiments, the integration of the exogenous sequence may result in the expression of the integrated sequence from an endogenous promoter sequence present on the patient's genomic DNA. For example, the administration of the vector system may result in supply of a functional gene product of the disease-associated gene to rectify the patient's mutation. In some embodiments, the administration of the vector system may result in integration of a cDNA sequence encoding a protein or a portion of the protein. In yet other embodiments, the administration of the vector system may result in integration of an exon sequence, an intron sequence, a transcriptional control sequence, a translational control sequence, or a non-coding sequence into the patient's genomic DNA. In some embodiments, the administration of the vector system may result in gene knockin in the patient.

Additional embodiments of the invention also encompass methods of treating the patient in a tissue-specific manner. In some embodiments, the method may comprise administering the vector system comprising a tissue-specific promoter as described herein to the patient. Non-limiting examples of suitable tissues for treatment by the methods include the immune system, neuron, muscle, pancreas, blood, kidney, bone, lung, skin, liver, and breast tissues.

The words “a”, “an” or “the” when used in conjunction with the term “comprising” in the claims and/or the specification may mean “one,” but each is also consistent with the meaning of “one or more,” “at least one,” and “one or more than one.” The use of “or” means “and/or” unless stated otherwise. The use of the term “including” and “containing,” as well as other forms, such as “includes,” “included,” “contains,” and “contained” is not limiting. All ranges given in the application encompass the endpoints unless stated otherwise.

### EXAMPLES

### Example 1

FIG. 1 shows a vector containing a nuclease system (e.g., CRISPR/Cas9) and a template, with target sequences located such that the template is released upon expression of the nuclease system, and the sequence expressing the nuclease is also cleaved. The guide RNA used to release the template and cleave the expression vector can be the same guide RNA that targets the desired genomic site.

The following plasmid constructs were made for this set of experiments. The plasmids used in the examples have a backbone containing an ampicillin resistance gene and a bacterial origin of replication.

Plasmid A (reporter): contains the following sequences in order
1. truncated CMV promoter
2. LacO
3. G5 target sequence
4. LacO
5. Luciferase.

In this particular plasmid, the above sequences are flanked by long terminal repeat (LTR) sequences for lentiviral expression. The LTRs, however, are not required for this experiment. The lacO sequences may be used to selectively regulate expression, as described in Example 3 below.

Plasmid B (template and guide RNA): contains the following sequences in order
1. G5 target sequence
2. template sequence containing a multiple cloning site (EcoRI, NotI, MluI) instead of wild-type G5 target sequence
3. G5 target sequence
4. U6 promoter
5. sequence encoding guide RNA G5 (single-guide RNA with truncated tracr having a total length of 103 nt).

Plasmid C (Cas9): contains the following sequences in order
1. CMV promoter
2. codon optimized Cas9 with three SV40 nuclear localization signals

In this system, the guide RNA targets a specific sequence, G5, in a particular human gene. The template in Plasmid B is homologous to the human gene target, except that the G5 target sequence was replaced with a multiple cloning site. Thus, when Plasmids B and C are both introduced into a human cell, guide RNA G5 and Cas9 should be co-expressed, leading to cleavage of genomic target DNA, and template DNA should also be released from Plasmid B. In a typical system, the Cas9-encoding sequence will also contain a G5 target sequence to allow self-inactivation of Cas9. In this experiment, however, reporter Plasmid A was added to monitor Cas9 activity.

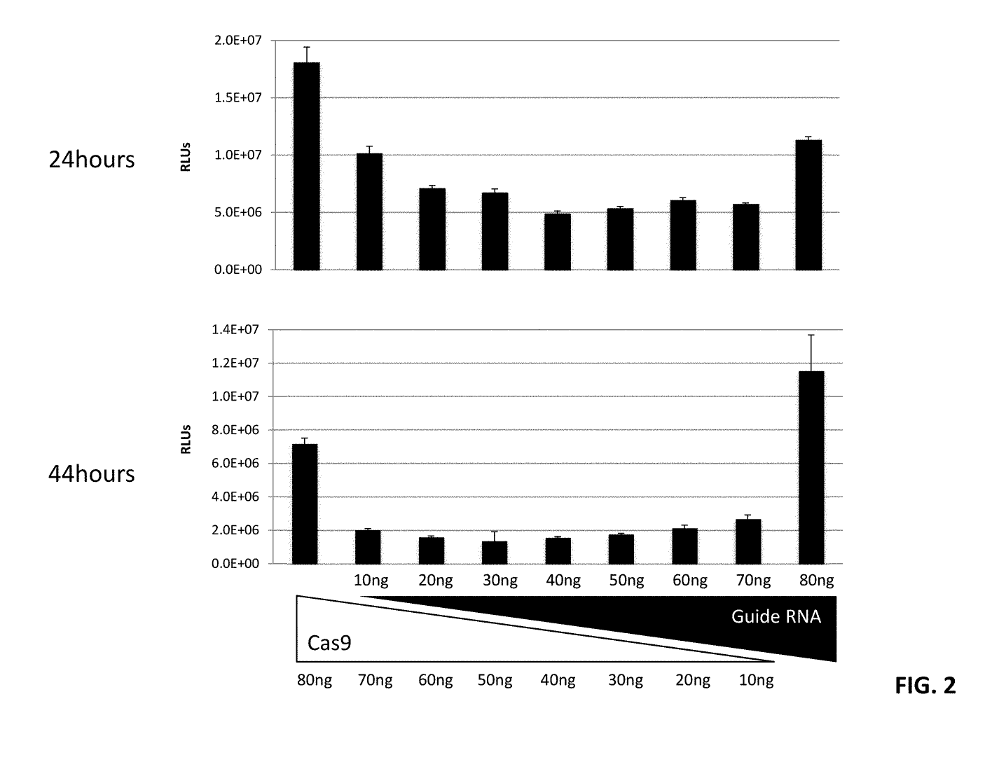

HEK293 cells were transfected with 10 ng of Plasmid A, 0 to 80 ng of Plasmid B, and 0 to 80 ng of Plasmid C, as shown in FIG. 2. Forty thousand cells per well were seeded in a 96-well poly-L-lysine coated plate, and incubated for 24 hours at 37° C./5% CO₂, then transfected with plasmids using Lipofectamine LTX in a total volume of 100 μL. Luciferase activity was measured at 24 hours and 44 hours after transfection (two separate sets of samples were prepared for each time point, n=8 for each experimental condition).

As shown in FIG. 2, samples without Plasmid B (i.e., no guide RNA) or Plasmid C (i.e., no Cas9) showed high luciferase activity, while samples with any combination and amount of Cas9 and guide RNA showed significant reduction in luciferase activity. The low level of luciferase activity at 24 hours in the rightmost lane (no Cas9 control) compared with the leftmost lane (no guide RNA control) was due to minor contamination during the experiment. This indicates that guide RNA and Cas9 produced from Plasmids B and C successfully cleaved reporter Plasmid A at the target sequence, resulting in loss of luciferase expression.

### Example 2

Additional testing was performed to determine whether the plasmid system released the template sequence from Plasmid B, and whether homologous recombination with genomic DNA occurred in the cells.

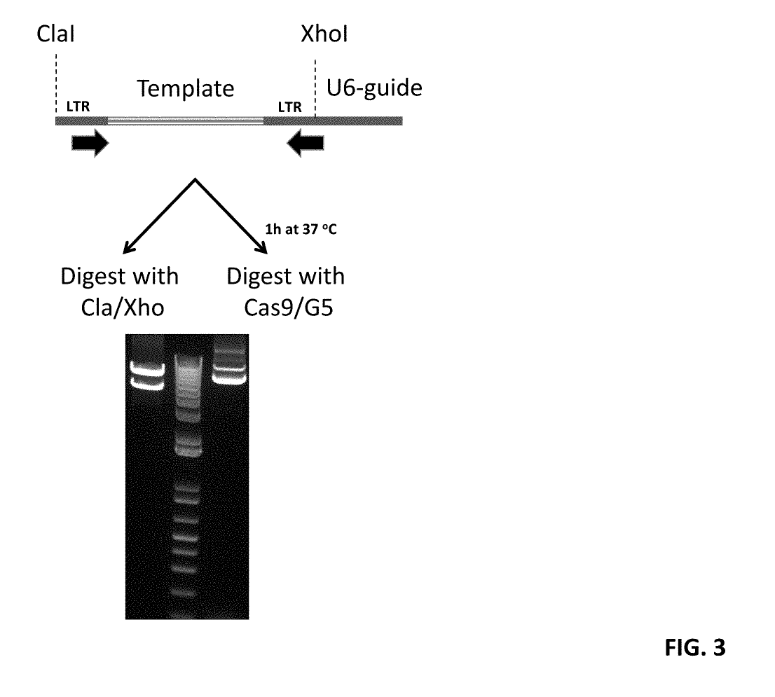

As a preliminary test of template release, Plasmid B was incubated for 1 hour at 37° C. with 1) Cas9 and guide RNA G5, or 2) ClaI and XhoI (Plasmid B contains ClaI and XhoI restriction sites adjacent to the target sequences). For the ClaI/XhoI digestion, a 50 μl reaction was prepared containing 1 μg of Plasmid B in a final volume of 42.5 μl, 5 μl of 10× CutSmart buffer (NEB), and 17 units of ClaI (1.7 μl) and 16 units of XhoI (0.8 μl). For Cas9/guide cleavage, 6.741 of crRNA (100 μM) and 3.75 μl of trRNA (100 μM) were heated at 95° C. for 2 min, respectively. The Cas9 master mix was made by adding 2.19 μl of Cas9 (10 mg/ml) in 1.38 μl of 5×CCE solution (with DTT). The trRNA was added to the Cas9 master mix and incubated at 37° C. for 5 min. The crRNA was subsequently added to the mixture of trRNA and Cas9 and incubated at 37° C. for 5 min to obtain the ribonucleoprotein complex of Cas9 and a sgRNA. The ribonucleoprotein complex was stored on ice until used. 1 μg of ribonucleoprotein complex was added to 1 μg of Plasmid B, in a final volume of 45 μl, to which was added 5 μl of 10× Cas9 buffer (NEB). FIG. 3 shows cleavage product under both sets of conditions, indicating that the template can be released from Plasmid B using Cas9.

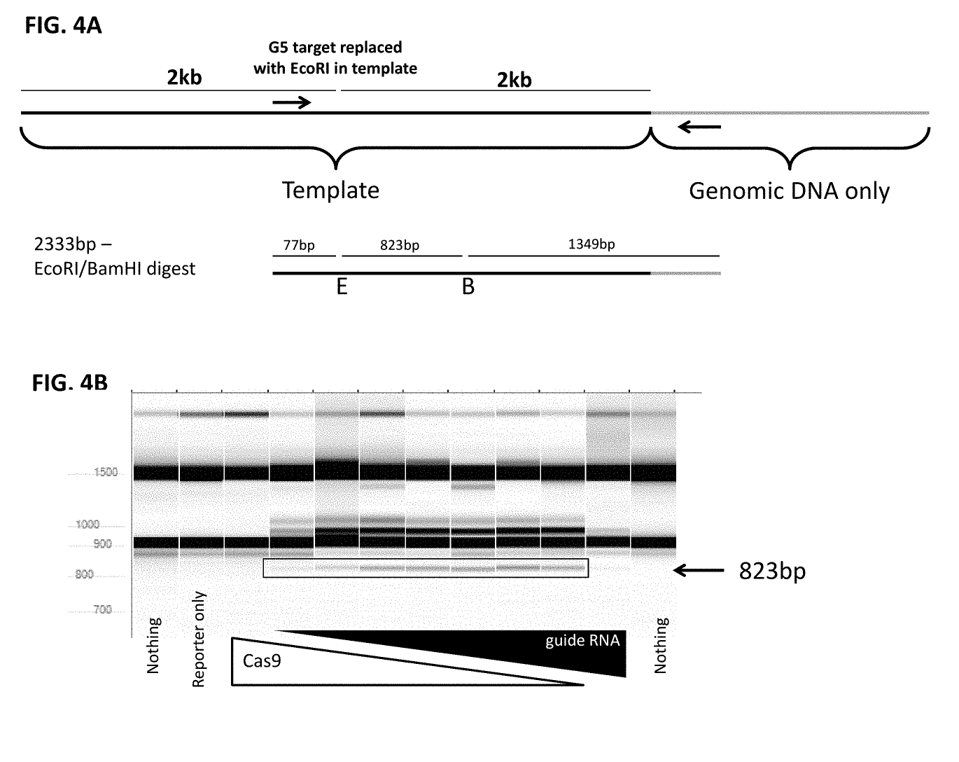

Furthermore, the samples from the 24-hour experiment in Example 1 were analyzed for homologous recombination products. Specifically, PCR primers were designed with one primer within the template sequence, and one primer within genomic DNA adjacent to the expected homologous recombination site (FIG. 4A). Standard PCR reactions were performed (35 cycles) to generate a 2333 bp product.

The 2333 bp amplification products were digested with EcoRI and BamHI for 1 hr at 37° C. Because the template sequence from Plasmid B contains an engineered EcoRI site not present in the corresponding genomic sequence, only recombination products should show the 823 bp dual-cleavage fragment upon treatment with EcoRI/BamHI. FIG. 4B shows that samples containing both Plasmids B and C have a fragment corresponding to the Plasmid B template sequence successfully inserted into the appropriate genomic location. The faint band at 823 bp in the second right lane was due to minor contamination during the experiment.

To further test the effect of Cas9 half-life on homologous recombination, a series of self-cleaving Cas9-expressing plasmids were designed containing a CMV promoter, target sequence for guide RNA G5, and a Cas9. The following Cas9 variants were tested in this construct:
1. Cas9 with 2×NLS and polyadenylation signal (PA)
2. Cas9 with 2×NLS and no PA
3. Cas9 with 2×NLS, PEST tag (degradation signal), and PA
4. Cas9 with 2×NLS, PEST tag, and no PA

In addition, CMV-Cas9 constructs without a self-cleavage target sequence were tested:
1. Cas9 with 3×NLS
2. Cas9 with 2×NLS

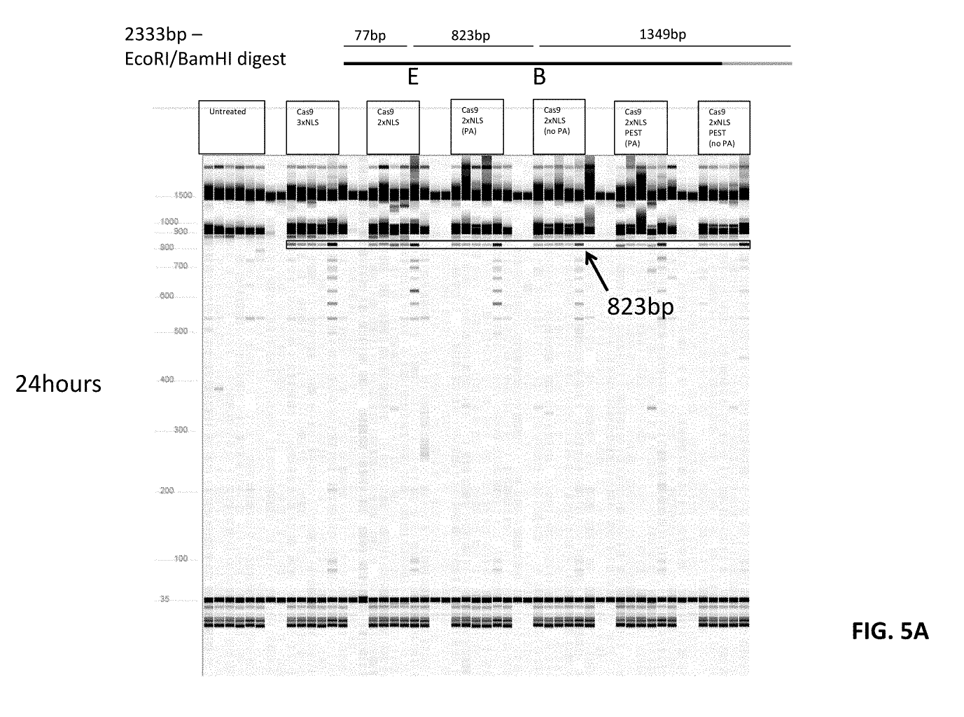

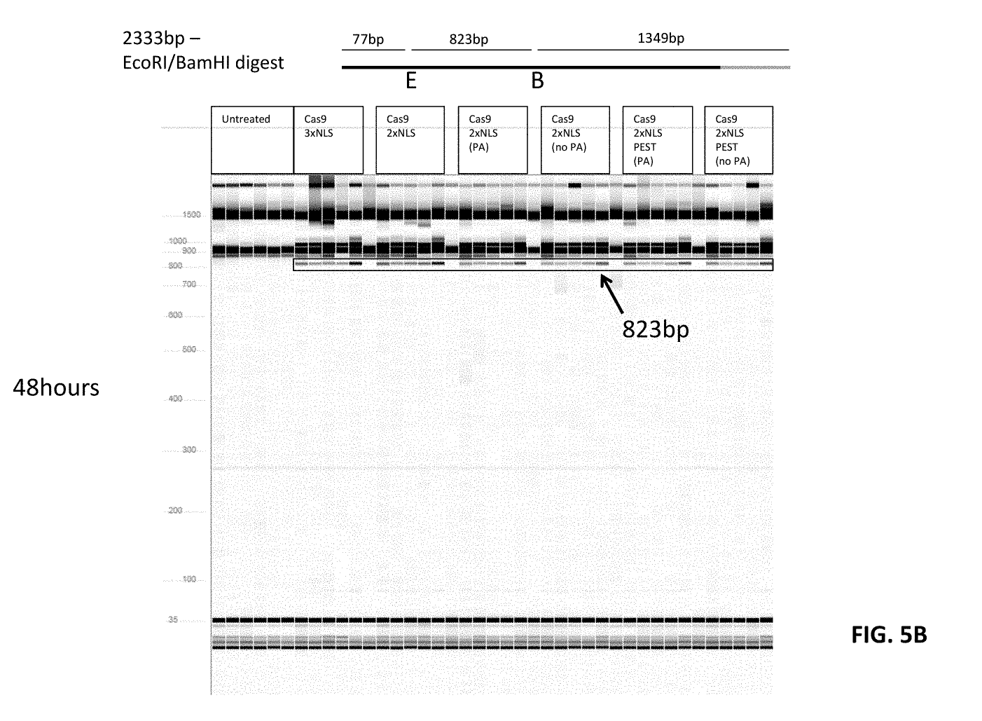

Each construct (10 ng) was transfected into HEK293 cells (forty thousand or twenty thousand cells per well) along with Plasmid B (80 ng), as described in Example 1. PCR followed by EcoRI/BamHI cleavage was performed as described above to identify homologous recombination products. FIGS. 5A and 5B show the results 24 hours and 48 hours after transfection, respectively. For each Cas9 variant tested and the untreated control, the first four lanes were loaded with samples from transfection with forty thousand cells per well, and the fifth lane was loaded with samples from transfection with twenty thousand cells per well. The results shown in FIG. 5 demonstrate that even in samples containing features that reduce Cas9 DNA, mRNA and/or protein half-life (i.e., self-cleaving vector, PEST tag, no PA), products corresponding to successful template insertion were observed. The cleavage signal increased 48 hours after transfection. In addition, the signal was higher in samples with fewer cells.

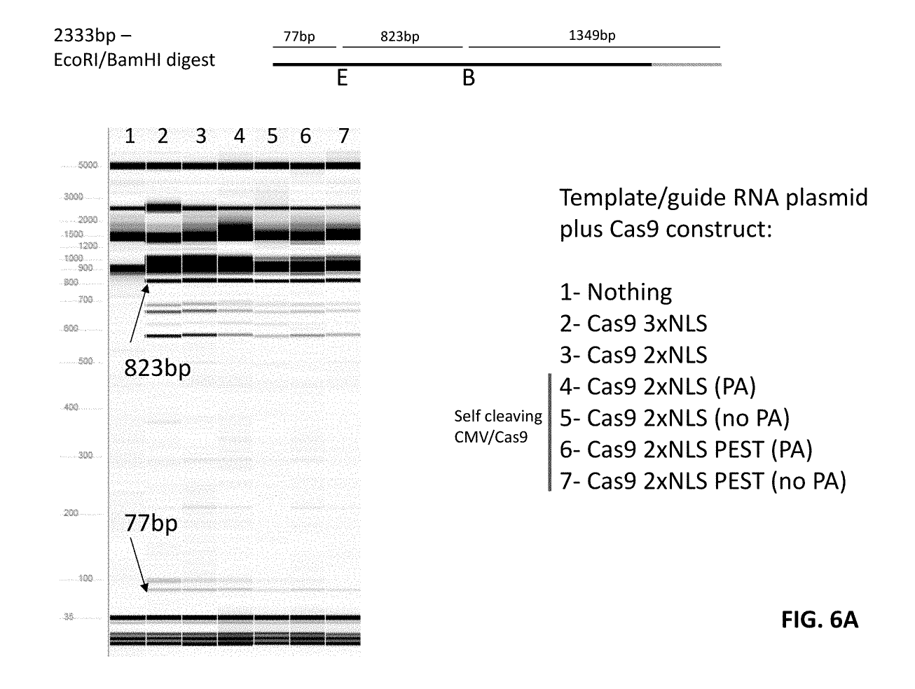

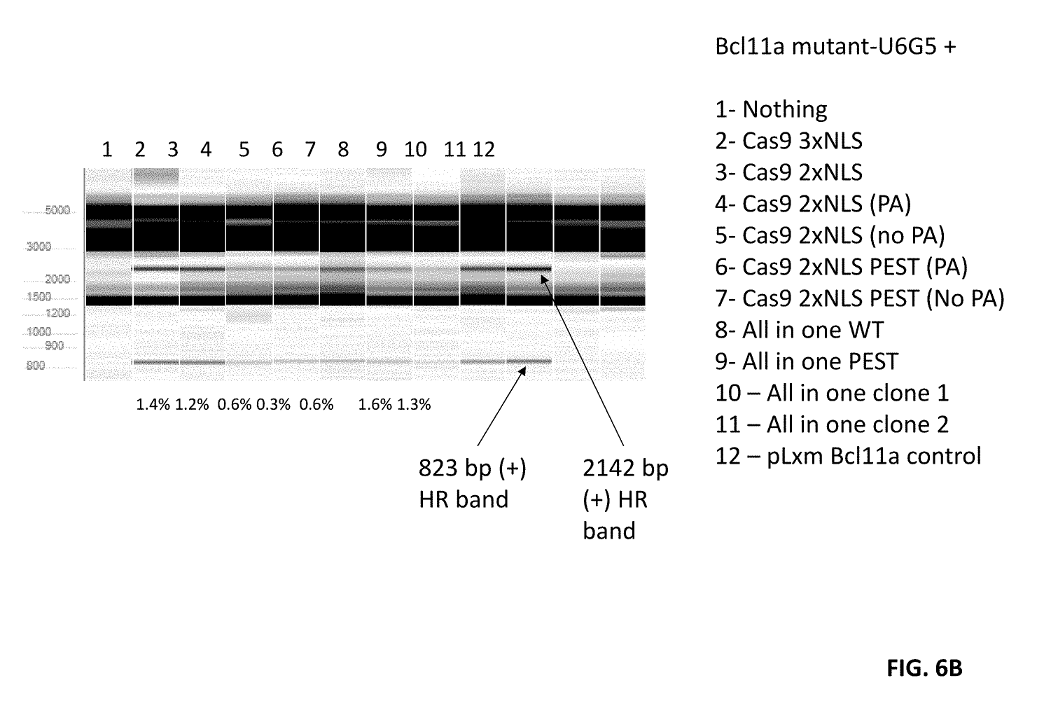

The above experiments with Cas9 variants were repeated with twenty thousand cells per well. Cleavage results from 24 hours after transfection were compared in FIG. 6A. Again, products corresponding to successful template insertion were observed. In addition, the following CMV-Cas9 constructs (90 ng) containing all components of the system on the same vector were introduced into HEK293 cells (twenty thousand cells per well) and tested under the experimental conditions described for FIG. 6A.
1. All in one WT (includes G5, template, G5, CMV, G5 target sequence, Cas9, U6-guide), all with PA
2. All in one PEST (includes G5, template, G5, CMV, G5 target sequence, Cas9-PEST, U6-guide)
   The homologous recombination was observed with these “all in one” plasmids.

PCR reactions and EcoRI/BamHI digestions were repeated for the samples shown in FIG. 6A and for the all-in-one constructs, but using primers only found in genomic DNA (i.e., not found in the template donor plasmid). These primers generate a 4299 bp amplicon, with the wild-type sequence resulting in 2951 bp and 1348 bp EcoRI/BamHI digestion products, and the homologous recombination product resulting in 2142 bp, 1351 bp, and 823 bp digestion products (FIG. 6B).

### Example 3

While two plasmids were used to deliver the CRISPR/Cas9 components in Example 1, all sequences can instead be contained on a single vector. With such a single-vector system, Cas9 and guide RNA sequences can be simultaneously expressed, and self-cleavage can occur during production of the vector. Thus, it is advantageous to prevent expression of Cas9 and/or guide RNA during vector production. To accomplish this, a LacI-KRAB repressor system was tested. Specifically, Plasmid A contains two lacO sites, inserted 57 bp apart, between the CMV promoter and the luciferase sequences. Cells were transfected with Plasmid A and a second plasmid expressing a LacI-KRAB fusion protein from a CMV promoter. Transfections were performed as described in Example 1, using 10 ng of Plasmid A and 80 ng of the LacI-KRAB construct.

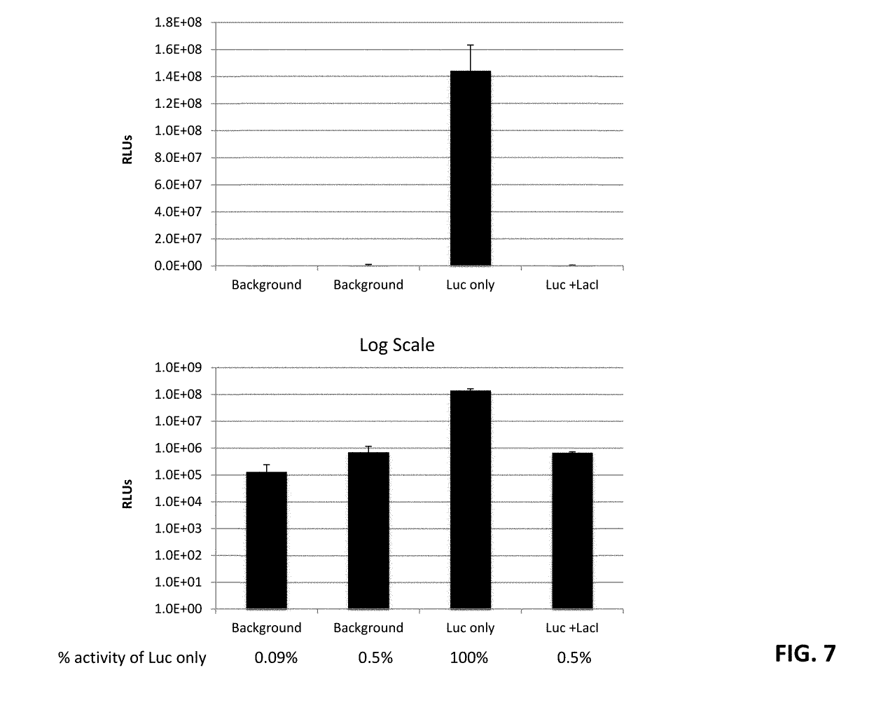

FIG. 7 shows that the presence of LacI-KRAB effectively eliminates luciferase expression. Accordingly, this repression system can be incorporated in a self-cleaving Cas9 expression construct to prevent destruction of the vector during production.

### Example 4

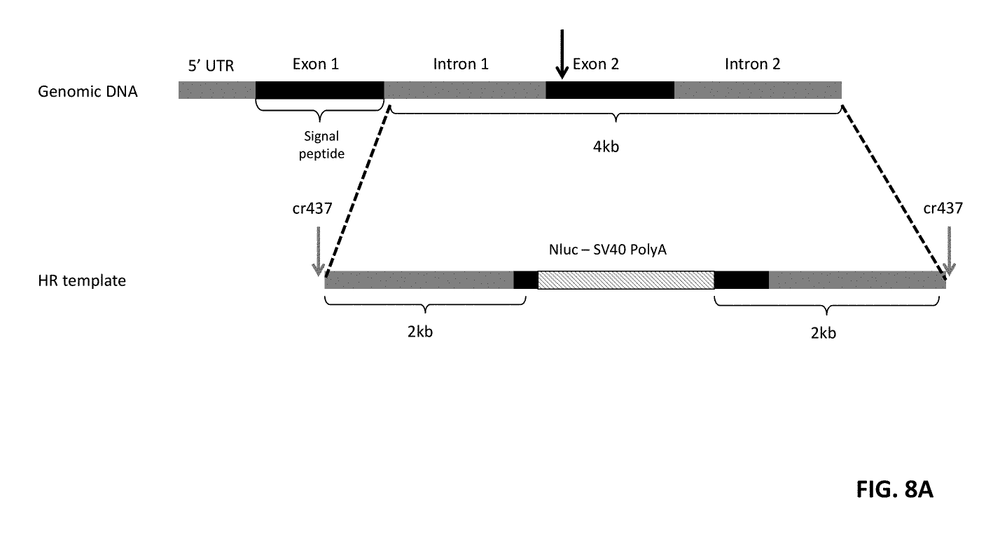

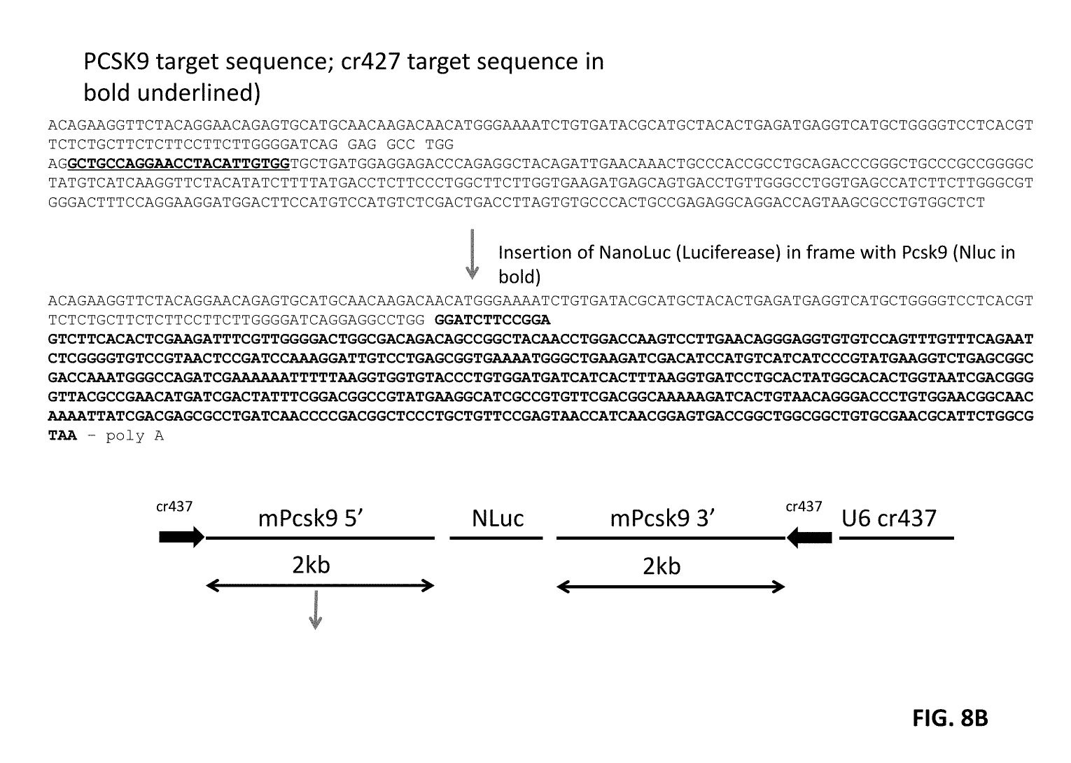

FIG. 8A shows an HR template that was designed for integrating a luciferase reporter gene (Nluc) into the mouse PCSK9 gene. PCSK9 encodes a protein secreted by hepatocytes in the liver, and also secreted by mouse liver cell lines such as the Hepa1.6 cells used herein. As designed, this HR template does not have a promoter for expressing Nluc and the ATG transcriptional start site was removed from the Nluc coding sequence. In this format, Nluc is not expressed from the template unless and until HR occurs between the template and the genomic PCSK9 gene, thereby inserting the Nluc sequence in-frame with the PCSK9 signal peptide, leading to secretion of the Nluc reporter gene into the culture media.

In addition to Plasmid C, the following plasmids were constructed and used in this experiment.

Plasmid D: contains the following sequences in order
1. cr437 (PSCK9) target sequence
2. Nluc template
3. cr437 (PSCK9) target sequence
4. U6 promoter
5. sequence encoding guide RNA cr437 (single-guide RNA with truncated tracr having a total length of 103 nt which targets the mouse PCSK9 gene).

Plasmid E: contains the following sequences in order
1. Nluc template
2. U6 promoter
3. sequence encoding guide RNA cr437 (single-guide RNA with truncated tracr having a total length of 103 nt which targets the mouse PCSK9 gene).

In this system, the cr437 guide RNA targets a specific sequence in the mouse PCSK9 gene. The template in Plasmids D and E comprise 2 kb homology arms that are homologous to PCSK9 and flank the Nluc reporter (FIG. 8A). The difference between Plasmids D and E is that Plasmid E does not contain the cr437 target sequence flanking the template, and therefore the template cannot be released by a Cas9/cr437 guide RNA complex. When Plasmids C and D are both introduced into a mouse cell, guide RNA c437 and Cas9 should be co-expressed, leading to cleavage of genomic PCSK9 DNA, and template DNA should also be released from Plasmid D. However, when Plasmids C and E are both introduced into a mouse cell, guide RNA c437 and Cas9 should be co-expressed, leading to cleavage of genomic PCSK9 DNA, but since Plasmid E does not contain the cr437 target sequence flanking the template, no template DNA should be released from Plasmid E. In a typical system, the Cas9-encoding sequence will also contain a cr437 target sequence to allow self-inactivation of Cas9. In this experiment, detection of Nluc activity in the culture media indicates that the template has been successfully integrated, in-frame, into the genomic PCSK9 gene by HR.

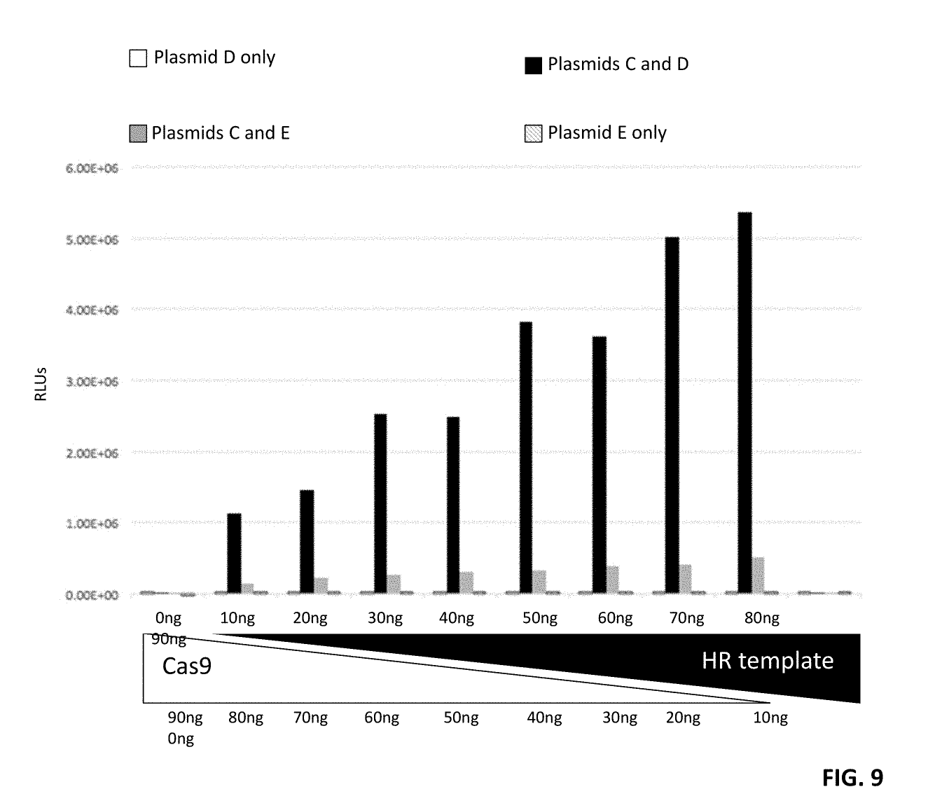

Hepa1.6 cells were transfected with Plasmid D alone, with Plasmid E alone, with Plasmids C and D, or with Plasmids C and E (ranging from 0 to 90 ng of each plasmid with a total of 90 ng per transfection, as shown in FIG. 9). Ten thousand cells per well were seeded in a 96-well plate, and incubated in DMEM with 10% FBS and 100 U/mL Pen-Strep, for 24 hours at 37° C./5% CO₂, then transfected with plasmids (total of 90 ng) using Lipofectamine 2000 in a total volume of 100 μL. Luciferase activity was measured at 24, 48, and 72 hours post-transfection using Promega's Nano-Glo Kit (two separate sets of samples were prepared for each time point, n=2 for each experimental condition).

As shown in FIG. 9, samples without Plasmid C (i.e., no Cas9) or without Plasmid D or Plasmid E (i.e., no template) showed no luciferase activity in the media at 72 hours post-transfection. Samples with any amount of Cas9 (from Plasmid C) and any amount of template (from Plasmid D or Plasmid E) showed significant luciferase activity, indicating that guide RNA and Cas9 produced from Plasmids C and D/E successfully cleaved the PCSK9 target sequence, resulting in HR and the in-frame insertion of Nluc into PCSK9. Substantial and dose dependent increases in luciferase activity were measured when Plasmids C and D were co-transfected with increasing amounts of Plasmid D (e.g., as compared to samples co-transfected with Plasmids C and E). This substantial improvement in HR efficiency indicates that use of vectors comprising templates with flanking target sequences (e.g., whereby template may be released via a Cas nuclease) increases HR efficiency. Similar results were observed at both 24 and 48 hours post-transfection for each condition (not shown).

As with Example 2, a PCR strategy was employed for analysis of HR products at the PCSK9 gene. The expected HR product where the template is inserted in-frame into PCSK9 is depicted in FIG. 8B.

Genomic DNA was purified from samples and sheared to an average size of 5 kb or 6 kb. An aliquot of 6 μg of gDNA was used for eighty cycles of linear amplification with a biotinylated oligonucleotide (Bio-mPC605; /5Biosg/AAGGAGGTTAGGCATGTCTC) (SEQ ID NO: 6), which anneals at a region upstream of the HR template. Amplified DNA was captured by magnetic Dynabeads C1 Streptavidin beads followed by three rounds of washes. Purified DNA/beads were used for a second linear amplification with a primer 32 nucleotides downstream of the cleavage site (dsmPC rev; GTGGGCAGTTTGTTCAATCTG) (SEQ ID NO: 7). ETDA was added to a final concentration of 7.5 mM prior to elution at 95° C. Eluted ssDNA was purified with Ampure XP beads followed by a sonication step to shear the DNA to around 300 nucleotides. A library kit for Illumina from Swift Biosciences (Accel-NGS 1S Plus DNA Library Kit for Illumina) was used to repair DNA ends, add adapters and to amplify the library. The resulting library was quantified by qPCR (KAPA Biosystems) and sequenced on an Illumina MiSeq instrument with pair-end 2×150 cycles.

Sequencing data from Read 2 (second primer) were analyzed to determine the percentage that contains HR product. In this experiment, around 2% of the reads contained luciferase sequence when using Plasmid D (e.g., wherein the template is released via a Cas9/cr437 guide RNA complex) in combination with a vector expressing Cas9 (e.g., Plasmid C). This result is consistent with the detection of secreted luciferase activity present in the culture media.

Sequences described in the above examples are listed as follows (polynucleotide sequences from 5′ to 3′):

Template flanked by G5 target sequences (underlined), with a partial G5 target sequence (underlined) inside the template and inserted by the EcoRI/NotI/MluI multiple cloning site

(SEQ ID NO: 8)

TTCGCGGCCGCACGCGT (bold)

(SEQ ID NO: 9)

AGGAGGTCATGATCCCCTTCTGGTCTTCCTTCAGTCTGTAAACCTCAGAA
CTTGTAGCTAATGCTAAACAAAAAAGCCACATTTATCAATGTGTACTTAA
AATCCTTAATTCAGACAACAGGAATATTTTGAGAATGAGTTCCCTATTCC
TCACTTGGTCAAAATGGAAGCAAATGTAAGAGAAGAATGACATTAAGGCA
CAATGCAGAGGCACTTCTGTTTGTCTTCTTTTATTTGAAAAGTATGCATA
TGTATTCTGTATTTATCTTTTGGCCAGTATGTTGGGCAAAGAAACATAAG
TGCTTACTTTACTGTCTTTATTAGTAGGAATATAACCTTCATATTCCTGT
GGTGACCTTATGTTAAATTAGGAGGAGTACCAGAGGCTAGAAATTATGAG
ATGTCCTACTTGAGCACAGGTGCAGCTAGGCAGGGCTCTCTCAATATTAT
TTCACCTAGCACATCTGGGAGTTACTCCAGATCTTCCCCCTCAATATTCA
GCCTGGGTAGGGTTGAAATAAATTTAACCTGAGTTCACTGGATTTTTGCA
CTTTATCAAAATCTGTTCCAATATTCTACACTCAAATTAAAATCTATTTT
TTGATTCTCTGTGGCTTTAAGTTCATTAAATGTAAAATTGGCAGCTTGCT
AAAGAAGGTCAGACTGATTAACTGTTTAAGACTTGTACATTTTCTGCTTC
AGTTTTATTAACTGGCAGCATCCTGGATGTTTTGTATTTTGTGATTTTTT
TTTTTTTTTTGATAGAGCAAGCATAAGATTTCACAAGCAGAGACTTACCA
ACTCTCTTTTCCCCTTTGGAAGCTTAAAAAATGATAGAAGCTGGTAAAGT
AGATGCTGGAGTATTTTAGTACAAAGTTAAAAAAAAAAGCAAACAGGAAA
GAAAGACATGTCTACCTTGTTATACCATCCGCTGGTGATTATGTGTGCAG
AAATAGTCTCATAATGAAGCATTTTGGAGCTCATTCAGAAAATTAGTCCA
CTTTGACAACATTAGGCGAAGTATTTCAAGTCTAAAGAAAGGACTTCTCA
GCCTTGCTCTGAAATGTGGTGTTTGCTTGACCATTCTGATTTTTATATCA
TAGATGCCACCAAGTGCAAACATGTTTAGAATATTATAGGCATTCCATTT
CTCAGAATAAAAAAAAAATGACTAATTGGCTTATTTTCTTAAGTACTCAA
AAGTATCCCATTTAGCTAATGTGTCTGAGAAATACTGCCCGTGCATTTGG
TATTTCTTTGATTTTGTGGCACTGCTGAGAGTGAGAGCAGAAAGGTTTTT
GGCAGTGTGAATTATGCTGCGACATGATTATTATTTAGATCCGTTTCATA
GGTGCATGCAGTCGTTTTCTTATTACAGCAGTGTAAATGTGGCACATTTT
TCATGTGACATAGTAGCTTTCTAATTTATGAAGCCATGTCTGTTTACTTA
GGAGTATATACATTCACACACAAAGGGTGTGTGTGTTTATTCACCTCTCC
TTTCATTCTTTGGCACAATGGACAACTTGGTGTATAGGAAAAAAGAAACA
AATTTGGTTTCTATCCACTTTTTTTTTTAACCAGTTTTTCTTGTAGTTAT
TATTTAAGCTTTCTTTATGTTCCCTGTGTTAACTATTTAAGTAGCATTCT
TTCTAAACTTACAAACCAGACACATTTGTTGCTGTGGGTGTGTGCATGGG
TATATGTGTGTGTGTGTGTTCTCTGGAGTTATGCAAGGAAGACTGTTTTC
TTTACATATGTGATGATTTGCCTCATTGACAAATTTGCTCTCTGGTTGAT
AACCTTCACATCCTTGTACTTTTTGTATGCTCACATTTTCTGGGTATTAT
ATAGAGAAGCCTAGAAACACTTTACATGATGTGGTGGGATGGCATGGGGT
TGAGATGTGCTTCTCCCCTTTCTGTCCTCTCTGGCACTCTAATAATTGTG
CTTTTGTTTCTCCAACCACAGCCGAGCCTCTTGAAGCCATTCTTACAGAT
GATGAACCAGACCACGGCCCGTTGGGAGCTCCAGAATTCGCGGCCGCACG
CGTCACCTGTGGGCAGTGCCAGATGAACTTCCCATTGGGGGACATTCTTA
TTTTTATCGAGCACAAACGGAAACAATGCAATGGCAGCCTCTGCTTAGAA
AAAGCTGTGGATAAGCCACCTTCCCCTTCACCAATCGAGATGAAAAAAGC
ATCCAATCCCGTGGAGGTTGGCATCCAGGTCACGCCAGAGGATGACGATT
GTTTATCAACGTCATCTAGAGGAATTTGCCCCAAACAGGAACACATAGCA
GGTAAATGAGAAGCAAGGAGAAAAGCTGTTTGCATGTTTTCTTTTCATTT
TCAGAGGTGCTGTAGCCAAGCAGTAAGGAGTTGTGAAGTGCTTTCTCTAT
TACTCTATGTGACTGTCCATGACAGCCCTGTAATGTTAAAATAATCATTT
CTGTTGCTTACGTCCAGAACACAGAAAAATAAATATTTTCCACCTCACTG
AATCAGATGTAGGCAGGATAGGTACACACATCAGACACCTTCTCTCTGGA
TCTGTCGATTTTGGATTTCTTTTCTTCCCCATCCCCACCTTCTCATTTTG
AAGTATTGAGCTTTACTACACCTAGTCCAGCTTCCATTGTCCATTTCCAG
CCTTGGTGACGTGTCAGAGGCAAAGTGGCCATATAGGCATTTGCAGTTCA
GCCAATGACTTGTTTGACTCAGAACATCTGGCCAGGCCTCCTTAGGGGTT
CAGCTCGTTCTCAAGGCTTCCCTGAAGTAGAGTGGGCTGGCAGGGTAGTT
GGAGGTGGTGGAAAGAGTTAACTGAGCTTCAGGGCTAGCCTTGGATCCAT
ATTGGCTGTCAGCCCGGATGGGGCTGTAATTAAACACAGCCCCGTGGTGG
GATGACACCATGACCTTGACTTTAAGATGCCATTTTCGACTGGCCAGGCC
AGAGTAGAGAGGGCAGTTGCTGAAGCGCACAGACATGCTTACTCGAAAAG
TTTAAGGGCATGTTGGAAATTTCAAAAGGTTGGTTTGACAGGAACGGCTG
CTCCCTGCAGCCTGCCTCCTCAGCTAAATGATAAATGCTTCTCTGTGCTC
TCTCTTGTCTCTGATGTGGTTTTGACAGATGTATCTTGATTTTGTTTGTG
GTTTACACAGCCACATGTCACCCTTACAAATGTCCAGTCCAGACTCCACT
GTTTCTGCTATAACACAATGTAAAAATTTTCTTGGAAAAATACACACACG
TATTCAACAGCCCTCCCTCCTTTGGTTAATTTTAGCAGGGAGGCAGCTAG
GTGTGTGGGTTTCTCGGCAGCTCAAGGGAAAAGGAATTAAAGGCTAGCAG
TGGGACTTAAATTCCCTTCTCTAAGTGATAAACAGTAACACTATATAGTG
ACCCTCAAAACATTTTTTGCTTGAGCATGTTAGACAAAAGTCAATGCAGA
TTCTGTGATGACAGACATGCCATGCCTGTTGGTGGATCGCTTTCTTCCAT
CTACCTACCACCCAGCTCCCGAAAGGCAAGAGGTTTGTTCAGTTTTAGGA
AAGGTAGTGCATATCATGAATTGATTCACTGGAACTTGTCTCTCCGACCT
AGTTTGACCACAAAGTTGAACCATAATAGGTCAGTGGTCTAGAGGGGATT
AAATGTCATATTATTTCTCCTCTCCCCCTCTAGAATTTGATCATTAAAAC
CAAACATGGCATTTTCTTTCTTTTTTTAGTGCTTTCTGTGATAGCACTCA
GATACTTTCCCTTTAGTGAAATGGGAAATCTGCTGCTAGGGAAGCTGCAT
TTGTGGAGTGTATTTCTTGAATCCACCACATTTACCTTATGTGACATGTA
GGTGAAGATTTTATCTCCCCTACCCCCCAGCAGGATGTGGGAATGACCAT
TTCCATGTGTTGTCTTGTGACTGGAAGGAAAATGAACAGAAGTGTAAGGC
ATGATTAATGAAGCAAGAGCAGGCGGAAGGGGATTTGTCGTCTTCGGAGA
TCCAAAGCCTTGCTAAATCACCAAATATGGAGTAACACTTGCGTGATGTA
ACATCGTATTTACATATCGAGCTGCTCGTTTAAAAGACAAAACACAGTGT
CTGTCAAGCAAGAATTAAAACCACACTTCTTACTGAGGTCCCAGAAGGGG
ATCATGACCTCCT

Truncated CMV (tCMV) inserted with a G5 target sequence (reverse orientation, bold) flanked by two LacO sites (underlined), shown with a start codon (ATG) at the end, which is under the control of tCMV

(SEQ ID NO: 10)

ATCGATAGCGGTTTGACTCACGGGGATTTCCAAGTCTCCACCCCATTGAC
GTCAATGGGAGTTTGTTTTGGCACCAAAATCAACGGGACTTTCCAAAATG
TCGTAACAACTCCGCCCCATTGACGCAAATGGGCGGTAGGCGTGTACGGT
GGGAGGTCTATATAAGCAGAGCTCTCTGGCTAATTGTGAGCGCTCACAAT
TCCCGTTGGGAGCTCCAGAAGGGGATCATGACCTCCTAATTGTGAGCGCT
CACAATTTAAATAGCCACCATG

Cas9 2×NLS

(SEQ ID NO: 11)

ATGGATAAGAAGTACTCAATCGGGCTGGATATCGGAACTAATTCCGTGGG
TTGGGCAGTGATCACGGATGAATACAAAGTGCCGTCCAAGAAGTTCAAGG
TCCTGGGGAACACCGATAGACACAGCATCAAGAAAAATCTCATCGGAGCC
CTGCTGTTTGACTCCGGCGAAACCGCAGAAGCGACCCGGCTCAAACGTAC
CGCGAGGCGACGCTACACCCGGCGGAAGAATCGCATCTGCTATCTGCAAG
AGATCTTTTCGAACGAAATGGCAAAGGTCGACGACAGCTTCTTCCACCGC
CTGGAAGAATCTTTCCTGGTGGAGGAGGACAAGAAGCATGAACGGCATCC
TATCTTTGGAAACATCGTCGACGAAGTGGCGTACCACGAAAAGTACCCGA
CCATCTACCATCTGCGGAAGAAGTTGGTTGACTCAACTGACAAGGCCGAC
CTCAGATTGATCTACTTGGCCCTCGCCCATATGATCAAATTCCGCGGACA
CTTCCTGATCGAAGGCGATCTGAACCCTGATAACTCCGACGTGGATAAGC
TTTTCATTCAACTGGTGCAGACCTACAACCAACTGTTCGAAGAAAACCCA
ATCAATGCTAGCGGCGTCGATGCCAAGGCCATCCTGTCCGCCCGGCTGTC
GAAGTCGCGGCGCCTCGAAAACCTGATCGCACAGCTGCCGGGAGAGAAAA
AGAACGGACTTTTCGGCAACTTGATCGCTCTCTCACTGGGACTCACTCCC
AATTTCAAGTCCAATTTTGACCTGGCCGAGGACGCGAAGCTGCAACTCTC
AAAGGACACCTACGACGACGACTTGGACAATTTGCTGGCACAAATTGGCG
ATCAGTACGCGGATCTGTTCCTTGCCGCTAAGAACCTTTCGGACGCAATC
TTGCTGTCCGATATCCTGCGCGTGAACACCGAAATAACCAAAGCGCCGCT
TAGCGCCTCGATGATTAAGCGGTACGACGAGCATCACCAGGATCTCACGC
TGCTCAAAGCGCTCGTGAGACAGCAACTGCCTGAAAAGTACAAGGAGATC
TTCTTCGACCAGTCCAAGAATGGGTACGCAGGGTACATCGATGGAGGCGC
TAGCCAGGAAGAGTTCTATAAGTTCATCAAGCCAATCCTGGAAAAGATGG
ACGGAACCGAAGAACTGCTGGTCAAGCTGAACAGGGAGGATCTGCTCCGG
AAACAGAGAACCTTTGACAACGGATCCATTCCCCACCAGATCCATCTGGG
TGAGCTGCACGCCATCTTGCGGCGCCAGGAGGACTTTTACCCATTCCTCA
AGGACAACCGGGAAAAGATCGAGAAAATTCTGACGTTCCGCATCCCGTAT
TACGTGGGCCCACTGGCGCGCGGCAATTCGCGCTTCGCGTGGATGACTAG
AAAATCAGAGGAAACCATCACTCCTTGGAATTTCGAGGAAGTTGTGGATA
AGGGAGCTTCGGCACAAAGCTTCATCGAACGAATGACCAACTTCGACAAG
AATCTCCCAAACGAGAAGGTGCTTCCTAAGCACAGCCTCCTTTACGAATA
CTTCACTGTCTACAACGAACTGACTAAAGTGAAATACGTTACTGAAGGAA
TGAGGAAGCCGGCCTTTCTGTCCGGAGAACAGAAGAAAGCAATTGTCGAT
CTGCTGTTCAAGACCAACCGCAAGGTGACCGTCAAGCAGCTTAAAGAGGA
CTACTTCAAGAAGATCGAGTGTTTCGACTCAGTGGAAATCAGCGGGGTGG
AGGACAGATTCAACGCTTCGCTGGGAACCTATCATGATCTCCTGAAGATC
ATCAAGGACAAGGACTTCCTTGACAACGAGGAGAACGAGGACATCCTGGA
AGATATCGTCCTGACCTTGACCCTTTTCGAGGATCGCGAGATGATCGAGG
AGAGGCTTAAGACCTACGCTCATCTCTTCGACGATAAGGTCATGAAACAA
CTCAAGCGCCGCCGGTACACTGGTTGGGGCCGCCTCTCCCGCAAGCTGAT
CAACGGTATTCGCGATAAACAGAGCGGTAAAACTATCCTGGATTTCCTCA
AATCGGATGGCTTCGCTAATCGTAACTTCATGCAATTGATCCACGACGAC
AGCCTGACCTTTAAGGAGGACATCCAAAAAGCACAAGTGTCCGGACAGGG
AGACTCACTCCATGAACACATCGCGAATCTGGCCGGTTCGCCGGCGATTA
AGAAGGGAATTCTGCAAACTGTGAAGGTGGTCGACGAGCTGGTGAAGGTC
ATGGGACGGCACAAACCGGAGAATATCGTGATTGAAATGGCCCGAGAAAA
CCAGACTACCCAGAAGGGCCAGAAAAACTCCCGCGAAAGGATGAAGCGGA
TCGAAGAAGGAATCAAGGAGCTGGGCAGCCAGATCCTGAAAGAGCACCCG
GTGGAAAACACGCAGCTGCAGAACGAGAAGCTCTACCTGTACTATTTGCA
AAATGGACGGGACATGTACGTGGACCAAGAGCTGGACATCAATCGGTTGT
CTGATTACGACGTGGACCACATCGTTCCACAGTCCTTTCTGAAGGATGAC
TCGATCGATAACAAGGTGTTGACTCGCAGCGACAAGAACAGAGGGAAGTC
AGATAATGTGCCATCGGAGGAGGTCGTGAAGAAGATGAAGAATTACTGGC
GGCAGCTCCTGAATGCGAAGCTGATTACCCAGAGAAAGTTTGACAATCTC
ACTAAAGCCGAGCGCGGCGGACTCTCAGAGCTGGATAAGGCTGGATTCAT
CAAACGGCAGCTGGTCGAGACTCGGCAGATTACCAAGCACGTGGCGCAGA
TCTTGGACTCCCGCATGAACACTAAATACGACGAGAACGATAAGCTCATC
CGGGAAGTGAAGGTGATTACCCTGAAAAGCAAACTTGTGTCGGACTTTCG
GAAGGACTTTCAGTTTTACAAAGTGAGAGAAATCAACAACTACCATCACG
CGCATGACGCATACCTCAACGCTGTGGTCGGTACCGCCCTGATCAAAAAG
TACCCTAAACTTGAATCGGAGTTTGTGTACGGAGACTACAAGGTCTACGA
CGTGAGGAAGATGATAGCCAAGTCCGAACAGGAAATCGGGAAAGCAACTG
CGAAATACTTCTTTTACTCAAACATCATGAACTTTTTCAAGACTGAAATT
ACGCTGGCCAATGGAGAAATCAGGAAGAGGCCACTGATCGAAACTAACGG
AGAAACGGGCGAAATCGTGTGGGACAAGGGCAGGGACTTCGCAACTGTTC
GCAAAGTGCTCTCTATGCCGCAAGTCAATATTGTGAAGAAAACCGAAGTG
CAAACCGGCGGATTTTCAAAGGAATCGATCCTCCCAAAGAGAAATAGCGA
CAAGCTCATTGCACGCAAGAAAGACTGGGACCCGAAGAAGTACGGAGGAT
TCGATTCGCCGACTGTCGCATACTCCGTCCTCGTGGTGGCCAAGGTGGAG
AAGGGAAAGAGCAAAAAGCTCAAATCCGTCAAAGAGCTGCTGGGGATTAC
CATCATGGAACGATCCTCGTTCGAGAAGAACCCGATTGATTTCCTCGAGG
CGAAGGGTTACAAGGAGGTGAAGAAGGATCTGATCATCAAACTCCCCAAG
TACTCACTGTTCGAACTGGAAAATGGTCGGAAGCGCATGCTGGCTTCGGC
CGGAGAACTCCAAAAAGGAAATGAGCTGGCCTTGCCTAGCAAGTACGTCA
ACTTCCTCTATCTTGCTTCGCACTACGAAAAACTCAAAGGGTCACCGGAA
GATAACGAACAGAAGCAGCTTTTCGTGGAGCAGCACAAGCATTATCTGGA
TGAAATCATCGAACAAATCTCCGAGTTTTCAAAGCGCGTGATCCTCGCCG
ACGCCAACCTCGACAAAGTCCTGTCGGCCTACAATAAGCATAGAGATAAG
CCGATCAGAGAACAGGCCGAGAACATTATCCACTTGTTCACCCTGACTAA
CCTGGGAGCCCCAGCCGCCTTCAAGTACTTCGATACTACTATCGATCGCA
AAAGATACACGTCCACCAAGGAAGTTCTGGACGCGACCCTGATCCACCAA
AGCATCACTGGACTCTACGAAACTAGGATCGATCTGTCGCAGCTGGGTGG
CGATGGCTCGGCTTACCCATACGACGTGCCTGACTACGCCTCGCTCGGAT
CGGGCTCCCCCAAAAAGAAACGGAAGGTGGACGGATCCCCGAAAAAGAAG
AGAAAGGTGGACTCCGGATGAGAATTCTCACGGCTTTCCGCCTGAGGTTG
AAGAGCAAGCCGCCGGTACATTGCCTATGTCCTGCGCACAAGAAAGCGGT
ATGGACCGGCACCCAGCCGCTTGTGCTTCAGCTCGCATCAACGTCTAAGG
CCGCGACTCTAGAGTCGGGGCGGCCGGCCGCTTCGAGCAGACATGATAAG
ATACATTGATGAGTTTGGACAAACCACAACTAGAATGCAGTGAAAAAAAT
GCTTTATTTGTGAAATTTGTGATGCTATTGCTTTATTTGTAACCATTATA
AGCTGCAATAAACAAGTTAACAACAACAATTGCATTCATTTTATGTTTCA
GGTTCAGGGGGAGGTGTGGGAGGTTTTTTAAAGCAAGTAAAACCTCTACA
AATGTGGTAAAATCGATAA

Cas9 2×NLS-PEST (PEST sequence underlined)

(SEQ ID NO: 12)

ATGGATAAGAAGTACTCAATCGGGCTGGATATCGGAACTAATTCCGTGGG
TTGGGCAGTGATCACGGATGAATACAAAGTGCCGTCCAAGAAGTTCAAGG
TCCTGGGGAACACCGATAGACACAGCATCAAGAAAAATCTCATCGGAGCC
CTGCTGTTTGACTCCGGCGAAACCGCAGAAGCGACCCGGCTCAAACGTAC
CGCGAGGCGACGCTACACCCGGCGGAAGAATCGCATCTGCTATCTGCAAG
AGATCTTTTCGAACGAAATGGCAAAGGTCGACGACAGCTTCTTCCACCGC
CTGGAAGAATCTTTCCTGGTGGAGGAGGACAAGAAGCATGAACGGCATCC
TATCTTTGGAAACATCGTCGACGAAGTGGCGTACCACGAAAAGTACCCGA
CCATCTACCATCTGCGGAAGAAGTTGGTTGACTCAACTGACAAGGCCGAC
CTCAGATTGATCTACTTGGCCCTCGCCCATATGATCAAATTCCGCGGACA
CTTCCTGATCGAAGGCGATCTGAACCCTGATAACTCCGACGTGGATAAGC
TTTTCATTCAACTGGTGCAGACCTACAACCAACTGTTCGAAGAAAACCCA
ATCAATGCTAGCGGCGTCGATGCCAAGGCCATCCTGTCCGCCCGGCTGTC
GAAGTCGCGGCGCCTCGAAAACCTGATCGCACAGCTGCCGGGAGAGAAAA
AGAACGGACTTTTCGGCAACTTGATCGCTCTCTCACTGGGACTCACTCCC
AATTTCAAGTCCAATTTTGACCTGGCCGAGGACGCGAAGCTGCAACTCTC
AAAGGACACCTACGACGACGACTTGGACAATTTGCTGGCACAAATTGGCG
ATCAGTACGCGGATCTGTTCCTTGCCGCTAAGAACCTTTCGGACGCAATC
TTGCTGTCCGATATCCTGCGCGTGAACACCGAAATAACCAAAGCGCCGCT
TAGCGCCTCGATGATTAAGCGGTACGACGAGCATCACCAGGATCTCACGC
TGCTCAAAGCGCTCGTGAGACAGCAACTGCCTGAAAAGTACAAGGAGATC
TTCTTCGACCAGTCCAAGAATGGGTACGCAGGGTACATCGATGGAGGCGC
TAGCCAGGAAGAGTTCTATAAGTTCATCAAGCCAATCCTGGAAAAGATGG
ACGGAACCGAAGAACTGCTGGTCAAGCTGAACAGGGAGGATCTGCTCCGG
AAACAGAGAACCTTTGACAACGGATCCATTCCCCACCAGATCCATCTGGG
TGAGCTGCACGCCATCTTGCGGCGCCAGGAGGACTTTTACCCATTCCTCA
AGGACAACCGGGAAAAGATCGAGAAAATTCTGACGTTCCGCATCCCGTAT
TACGTGGGCCCACTGGCGCGCGGCAATTCGCGCTTCGCGTGGATGACTAG
AAAATCAGAGGAAACCATCACTCCTTGGAATTTCGAGGAAGTTGTGGATA
AGGGAGCTTCGGCACAAAGCTTCATCGAACGAATGACCAACTTCGACAAG
AATCTCCCAAACGAGAAGGTGCTTCCTAAGCACAGCCTCCTTTACGAATA
CTTCACTGTCTACAACGAACTGACTAAAGTGAAATACGTTACTGAAGGAA
TGAGGAAGCCGGCCTTTCTGTCCGGAGAACAGAAGAAAGCAATTGTCGAT
CTGCTGTTCAAGACCAACCGCAAGGTGACCGTCAAGCAGCTTAAAGAGGA
CTACTTCAAGAAGATCGAGTGTTTCGACTCAGTGGAAATCAGCGGGGTGG
AGGACAGATTCAACGCTTCGCTGGGAACCTATCATGATCTCCTGAAGATC
ATCAAGGACAAGGACTTCCTTGACAACGAGGAGAACGAGGACATCCTGGA
AGATATCGTCCTGACCTTGACCCTTTTCGAGGATCGCGAGATGATCGAGG
AGAGGCTTAAGACCTACGCTCATCTCTTCGACGATAAGGTCATGAAACAA
CTCAAGCGCCGCCGGTACACTGGTTGGGGCCGCCTCTCCCGCAAGCTGAT
CAACGGTATTCGCGATAAACAGAGCGGTAAAACTATCCTGGATTTCCTCA
AATCGGATGGCTTCGCTAATCGTAACTTCATGCAATTGATCCACGACGAC
AGCCTGACCTTTAAGGAGGACATCCAAAAAGCACAAGTGTCCGGACAGGG
AGACTCACTCCATGAACACATCGCGAATCTGGCCGGTTCGCCGGCGATTA
AGAAGGGAATTCTGCAAACTGTGAAGGTGGTCGACGAGCTGGTGAAGGTC
ATGGGACGGCACAAACCGGAGAATATCGTGATTGAAATGGCCCGAGAAAA
CCAGACTACCCAGAAGGGCCAGAAAAACTCCCGCGAAAGGATGAAGCGGA
TCGAAGAAGGAATCAAGGAGCTGGGCAGCCAGATCCTGAAAGAGCACCCG
GTGGAAAACACGCAGCTGCAGAACGAGAAGCTCTACCTGTACTATTTGCA
AAATGGACGGGACATGTACGTGGACCAAGAGCTGGACATCAATCGGTTGT
CTGATTACGACGTGGACCACATCGTTCCACAGTCCTTTCTGAAGGATGAC
TCGATCGATAACAAGGTGTTGACTCGCAGCGACAAGAACAGAGGGAAGTC
AGATAATGTGCCATCGGAGGAGGTCGTGAAGAAGATGAAGAATTACTGGC
GGCAGCTCCTGAATGCGAAGCTGATTACCCAGAGAAAGTTTGACAATCTC
ACTAAAGCCGAGCGCGGCGGACTCTCAGAGCTGGATAAGGCTGGATTCAT
CAAACGGCAGCTGGTCGAGACTCGGCAGATTACCAAGCACGTGGCGCAGA
TCTTGGACTCCCGCATGAACACTAAATACGACGAGAACGATAAGCTCATC
CGGGAAGTGAAGGTGATTACCCTGAAAAGCAAACTTGTGTCGGACTTTCG
GAAGGACTTTCAGTTTTACAAAGTGAGAGAAATCAACAACTACCATCACG
CGCATGACGCATACCTCAACGCTGTGGTCGGTACCGCCCTGATCAAAAAG
TACCCTAAACTTGAATCGGAGTTTGTGTACGGAGACTACAAGGTCTACGA
CGTGAGGAAGATGATAGCCAAGTCCGAACAGGAAATCGGGAAAGCAACTG
CGAAATACTTCTTTTACTCAAACATCATGAACTTTTTCAAGACTGAAATT
ACGCTGGCCAATGGAGAAATCAGGAAGAGGCCACTGATCGAAACTAACGG
AGAAACGGGCGAAATCGTGTGGGACAAGGGCAGGGACTTCGCAACTGTTC
GCAAAGTGCTCTCTATGCCGCAAGTCAATATTGTGAAGAAAACCGAAGTG
CAAACCGGCGGATTTTCAAAGGAATCGATCCTCCCAAAGAGAAATAGCGA
CAAGCTCATTGCACGCAAGAAAGACTGGGACCCGAAGAAGTACGGAGGAT
TCGATTCGCCGACTGTCGCATACTCCGTCCTCGTGGTGGCCAAGGTGGAG
AAGGGAAAGAGCAAAAAGCTCAAATCCGTCAAAGAGCTGCTGGGGATTAC
CATCATGGAACGATCCTCGTTCGAGAAGAACCCGATTGATTTCCTCGAGG
CGAAGGGTTACAAGGAGGTGAAGAAGGATCTGATCATCAAACTCCCCAAG
TACTCACTGTTCGAACTGGAAAATGGTCGGAAGCGCATGCTGGCTTCGGC
CGGAGAACTCCAAAAAGGAAATGAGCTGGCCTTGCCTAGCAAGTACGTCA
ACTTCCTCTATCTTGCTTCGCACTACGAAAAACTCAAAGGGTCACCGGAA
GATAACGAACAGAAGCAGCTTTTCGTGGAGCAGCACAAGCATTATCTGGA
TGAAATCATCGAACAAATCTCCGAGTTTTCAAAGCGCGTGATCCTCGCCG
ACGCCAACCTCGACAAAGTCCTGTCGGCCTACAATAAGCATAGAGATAAG
CCGATCAGAGAACAGGCCGAGAACATTATCCACTTGTTCACCCTGACTAA
CCTGGGAGCCCCAGCCGCCTTCAAGTACTTCGATACTACTATCGATCGCA
AAAGATACACGTCCACCAAGGAAGTTCTGGACGCGACCCTGATCCACCAA
AGCATCACTGGACTCTACGAAACTAGGATCGATCTGTCGCAGCTGGGTGG
CGATGGCTCGGCTTACCCATACGACGTGCCTGACTACGCCTCGCTCGGAT
CGGGCTCCCCCAAAAAGAAACGGAAGGTGGACGGATCCCCGAAAAAGAAG
AGAAAGGTGGACTCCGGGAATTCTCACGGCTTTCCGCCTGAGGTTGAAGA
GCAAGCCGCCGGTACATTGCCTATGTCCTGCGCACAAGAAAGCGGTATGG
ACCGGCACCCAGCCGCTTGTGCTTCAGCTCGCATCAACGTCTAA

U6 G5 sgRNA (sgRNA sequence bold with G5 targeting sequence underlined)

(SEQ ID NO: 13)

GGGCCTATTTTCCCATGATTCCTTCATATTTGCATATACGATACAAGGCT
GTTAGAGAGATAATTGGAATTAATTTGACTGTAAACACAAAGATATTAGT
ACAAAATACGTGACGTAGAAAGTAATAATTTCTTGGGTAGTTTGCAGTTT
TAAAATTATGTTTTAAAATGGACTATCATATGCTTACCGTAACTTGAAAG
TATTTCGATTTCTTGGCTTTATATATCTTGTGGAAAGGACGAAACACCAG
GAGGTCATGATCCCCTTCGTTTTAGAGCTAGAAATAGCAAGTTAAAATAA
GGCTAGTCCGTTATCAACTTGAAAAAGTGGCACCGAGTCGGTGCTTTTTT
T

LacI-KRAB (KRAB domain underlined)

(SEQ ID NO: 14)

ATGAAGCCTGTGACCCTGTACGACGTGGCCGAGTACGCCGGAGTGAGTTA
TCAGACTGTGTCCCGGGTCGTGAATCAGGCCTCTCACGTGAGCGCTAAAA
CCCGCGAGAAAGTGGAGGCCGCAATGGCCGAACTGAACTATATCCCAAAC
CGCGTGGCCCAGCAGCTGGCAGGCAAGCAGAGCCTGCTGATCGGCGTGGC
TACCTCTAGCCTCGCATTGCACGCCCCGTCTCAGATCGTGGCCGCCATCA
AGTCCCGCGCTGATCAGCTGGGAGCTTCTGTCGTGGTGAGCATGGTCGAA
CGCTCCGGAGTGGAGGCCTGCAAGGCAGCCGTCCATAACCTCCTCGCCCA
GCGGGTTTCCGGCCTGATTATCAACTATCCACTGGATGACCAAGATGCCA
TCGCTGTCGAGGCGGCATGTACTAACGTGCCAGCCTTGTTTCTGGATGTG
AGCGACCAGACCCCTATCAACAGCATCATCTTCTCTCACGAGGACGGCAC
TAGGCTGGGTGTTGAGCACCTGGTCGCCCTGGGTCACCAGCAGATTGCCC
TCTTGGCCGGCCCACTGTCTAGTGTGAGTGCGAGACTGCGTTTGGCCGGT
TGGCACAAGTACCTGACACGCAACCAGATCCAGCCCATCGCGGAGCGTGA
GGGAGACTGGAGTGCCATGTCCGGCTTCCAGCAGACCATGCAGATGCTGA
ACGAAGGAATTGTGCCAACCGCCATGCTCGTGGCAAATGATCAGATGGCC
CTGGGAGCCATGAGGGCAATCACTGAGTCTGGCCTCCGTGTGGGTGCCGA
TATCAGTGTTGTGGGCTACGACGATACTGAGGACTCTAGTTGTTATATCC
CTCCCCTCACCACCATCAAACAGGACTTCAGGCTCCTGGGCCAAACCTCC
GTCGACCGGTTGCTGCAGCTCAGCCAGGGCCAGGCCGTCAAGGGAAACCA
GCTCCTGCCAGTTTCCTTGGTGAAGCGTAAAACAACCCTGGCACCAAATA
CTCAGACAGCGTCTCCTCGCGCGCTCGCGGATTCTCTGATGCAGCTCGCA
CGTCAGGTGAGCAGGTTGGAGTCCGGCCAACCGAAGAAGAAGAGAAAGGT
CGACGGAGGTGGCGCTCTGTCCCCTCAGCACAGTGCTGTGACACAGGGCA
GCATCATCAAGAACAAGGAGGGCATGGACGCCAAGAGCCTCACTGCCTGG
TCCCGTACCCTGGTGACTTTCAAGGACGTGTTCGTTGACTTTACCCGGGA
GGAGTGGAAGCTGCTGGATACCGCCCAGCAGATTGTCTATAGAAACGTCA
TGCTGGAGAACTACAAGAATCTCGTGTCCCTCGGATACCAGCTGACAAAA
CCTGACGTGATCCTGCGCCTCGAGAAGGGTGAAGAACCTTGGCTGGTGGA
AAGGGAAATTCACCAGGAGACCCACCCCGATTCCGAGACCGCCTTCGAGA
TCAAAAGCAGTGTGTAATGA

Nluc HR template for integration into PCSK9 (cr437 target sequence bold and underlined; Nluc sequence underlined; poly-A in bold)

(SEQ ID NO: 15)

GCTGCCAGGAACCTACATTGTGGGAGGAATAACTCTATCCATCAAAGTAA
TGCCCTGGGCAAGATGCTTCCTCTCCCCCTTTAGCAGTGAAGTGTAGGCA
CTGAAGGCCATTATATCATCACCCTTTCAGGCCTAGAAATCTTTTTCGGC
TCTAACATAGCAGAGCCATTTGATTCACTGTCTGATGGTCATAACACATT
TGCCTCTCAAACCCTATCTTCTGTCCTAACCCCCAAGCTGCTCAGCACTG
GTTACCATCGGAAGGTTTGGCATTTTGATTTTATGCTGTTTGATTACAGT
CTCTTTATGCCATCGAGCCCTGAACTGAAGGGATTTAGCAGGTTTTAAAC
AAGTCCTGGCCAGCGTGTCCCACTCATGGGGTATTAGGTGGTCTGCTTCA
GCCGTCCCTTTCAACAATTCCAAAGCCATATGGAGATATAGCTTCAGAAG
AGGGCATGGCATGTTTAAAACCCCCAAGTGTCGTATAGGGAAGGGAACAG
GCTCATCCTCTGTGTGTATTCCTCACTGAGGAAAAGCATCGTCAACTCTT
CGTGATGGTGGTGGATTCAAGGATTGAAGGGGATGGAAATACAAGAGGCA
AGGAGGTTAGGCATGTCTCAGGATCCTTCTTTTTGAGCTAACAGAACCTC
CCAGGATAATGCAAATGCATCAGCCCGTAGGGGTGCAGAGGAAGGGCTAG
TAGGGTGCAGAGGAAGGGCTAGTAGGGGTGCAGAGGAAGGGCTAGTAGGG
TGCAGAGGAAGGGCTAGTAGAGGTGCAGAGGAAGGGCTAGTAGGGGTGCA
GAGGAAGGGCTAGTAGGGTGCAGAGGAAGGGCTAGTAGGGTGCAGAGGAA
GGGCTAGTAGGGGTGCAGAGGAAGGGCTAGTAGGGGTGCAGAGAAAGGGC
TAGTAGGGTGCAGAGGAAGGGCTAGTAGGGTGCAGAGGAAGGGCTAGTAG
GGGTGCAGAGGAAGGGCTAGTAGGGTGCAGAGGAAGGGCTAGTAGGGTGC
AGAGGAAGGGCTAGTAGGGTGCAGAGGAAGGGCTAGTAGGGGTGCAGAGG
AAGGGCTAGTAGGGTGCAGAGAAAGGGCTAGTAGGGTGCAGAGGAAGGGC
TAGTAGGGTGCAGAGGAAGGGCTAGTAGGGGTGCAGAGGAAGGGCTAGTA
AAGTGCAGAGGAAGGGCTAGTAGAGGATGCTCTGTCTTCTGAATCATTGG
AAGAATCAGAAGACTGGGAATGGGGTGAGGGGAGCTGAAGGCTTCAGGCA
AGGCTTGCCTACTTCTGTCTCTCTGAAGGGTCTATCTGGTGCTTTCTCTC
TGTGCTTAGGGTAGGGGTGGTTTGCAAAGCCTGAATAGCTAAGGTGATCA
GATTAAAAGGGGCTGGACATTGAATGGGCCCACCTCTCCCCGCCCATGAA
CTTGTTTAAAATAACACAAAACACCTTTCCATTGCTTTATGTGTAATGTG
CCCTATGGTGGCAGTCAGGAGCAGTATGTCCATGTATTCTGACAGGCTAT
AGAGATCTGCTTTTTGCCCCTTCCACCATGCTTTGACCCCTCTGCACAAT
AGGCACATTGTAGTCTTTTCTTTTGTTTTGCTTTGCACCCATGATTACCC
TGGTGTCCTGGTGTGGGCTCCCATGTGTGTACCAGGACTACATACCTCTC
ATTAGATTCCCTCTGTTTTGCTCAGGCCCTGTTTGGGTACCACACGTTTC
AATCCACCAATGATTGGTGCAACTCAAGATTCAACAAGGCCAGGGCCTCT
ATGCTCCATAAGAACCTTTTTATTGGAGTTCTGTGGAGAGTTTATTTGGA
TAGTTCAGGGTTCAAAGCATGGGCAGAGAAACAGTGAAAAATATACACAT
TATTTATGATTATTCTCACCAGATGTACTCAGGAGACAGAAGGTTCTACA
GGAACAGAGTGCATGCAACAAGACAACATGGGAAAATCTGTGATACGCAT
GCTACACTGAGATGAGGTCATGCTGGGGTCCTCACGTTCTCTGCTTCTCT
TCCTTCTTGGGGATCAGGAGGCCTGGGGATCTTCCGGAGTCTTCACACTC
GAAGATTTCGTTGGGGACTGGCGACAGACAGCCGGCTACAACCTGGACCA
AGTCCTTGAACAGGGAGGTGTGTCCAGTTTGTTTCAGAATCTCGGGGTGT
CCGTAACTCCGATCCAAAGGATTGTCCTGAGCGGTGAAAATGGGCTGAAG
ATCGACATCCATGTCATCATCCCGTATGAAGGTCTGAGCGGCGACCAAAT
GGGCCAGATCGAAAAAATTTTTAAGGTGGTGTACCCTGTGGATGATCATC
ACTTTAAGGTGATCCTGCACTATGGCACACTGGTAATCGACGGGGTTACG
CCGAACATGATCGACTATTTCGGACGGCCGTATGAAGGCATCGCCGTGTT
CGACGGCAAAAAGATCACTGTAACAGGGACCCTGTGGAACGGCAACAAAA
TTATCGACGAGCGCCTGATCAACCCCGACGGCTCCCTGCTGTTCCGAGTA
ACCATCAACGGAGTGACCGGCTGGCGGCTGTGCGAACGCATTCTGGCGAA
TTCTCACGGCTTTCCGCCTGAGGTTGAAGAGCAAGCCGCCGGTACATTGC
CTATGTCCTGCGCACAAGAAAGCGGTATGGACCGGCACCCAGCCGCTTGT
GCTTCAGCTCGCATCAACGTCTAAGGCCGCGACTCTAGAGTCGGGGCGGC
CGGCCGCTTCGAGCAGACATGATAAGATACATTGATGAGTTTGGACAAAC
CACAACTAGAATGCAGTGAAAAAAATGCTTTATTTGTGAAATTTGTGATG
CTATTGCTTTATTTGTAACCATTATAAGCTGCAATAAACAAGTTAACAAC
AACAATTGCATTCATTTTATGTTTCAGGTTCAGGGGGAGGTGTGGGAGGT
TTTTTAAAGCAAGTAAAACCTCTACAAATGTGGTAGGCGCGCCTGTGGTG
CTGATGGAGGAGACCCAGAGGCTACAGATTGAACAAACTGCCCACCGCCT
GCAGACCCGGGCTGCCCGCCGGGGCTATGTCATCAAGGTTCTACATATCT
TTTATGACCTCTTCCCTGGCTTCTTGGTGAAGATGAGCAGTGACCTGTTG
GGCCTGGTGAGCCATCTTCTTGGGCGTGGGACTTTCCAGGAAGGATGGAC
TTCCATGTCCATGTCTCGACTGACCTTAGTGTGCCCACTGCCGAGAGGCA
GGACCAGTAAGCGCCTGTGGCTCTTGGTTCCCTGAATAACTAACTGCCTA
CTTAACTTGGCCACATCCCCATGCTGTGTCTACAATCATAGGAGGACAGA
GGGGATCACAAGGCAGCTAGCAGCAGAGCCCCTGCCTGCCAGTATACGTT
TCTGGTTTGTCTACTGCCTGTGAAAACCTGCAGGGACAAGGCCTGGGGAT
GCTGGTGACAAAGGTGTCAAATATGTCAGATTCTTCTCGTTTGGGACAAG
GTAGTGCTTCTCCATAACTCCTCTGAATGTTGCCTTTCTTTGCTAAGAAG
GTAAAAGGGGTACAGACTATCACCTGCCCTCCCTGTCTTCTCCCCATCTT
GGCACTCCAGGTTCTCACCTCTCTTCTCCACTGGGTACTCTCAGCCCCCT
GCACACCCTTAGGTCCCAGGGCTCAGGGCCTTGCCCAGGCCTCCAGTGTG
CATTGCACATGCACCTGGTCTTCTGGCCTCAGGTTCCTGCTCAGGGTTTA
AACTGACTTAAGATCTTGTTACTAAATGACAGTGGGGCATGGGCCATGCC
ACGGAGCAGGAGGACATCAAATCAGTGCCTCCCATCCATGCACTCTGCAC
TTTACCAAGCATCGCCTGTGACACAGCCTTGAACCTTTCCATCAAGCTTA
CGGCAAAGGTGGAGACTGGATGGATGGTTGATGCCAGAGCAGTTAGTTGG
TGCTGTGTGCTCAGTGCAGTGGGGAATGAGTAAGACACCTGAAGTGCCAG
GGGGCCGGCAGGTGCCCGCTGGTCAGGGCAACAAGCTCTGTACAGGGGGC
CATAGGATTTGCTCTAGGAACTTGAGCCCGGAGTCTCAAAGGGTGCACTG
GCCTCAGCTCAGTGTCCCACTAGTTTGTTTAGTTTAGAGCAGATGCCACT
CTCTCCCACGATTATCCGTAAGCCAGATGGGGTGATGGGAGCCATCTTTT
GAGGACTAGTGGAGACTGTGGAATATTTTTTTGAATAGGGATAGGTTGAG
ACAGTGTCTTGTTTTGTAGTCCAAGCTGGCCTCTCACTCGTGATAACCCC
ACCCCTGCTTCTGGGATGCTTGTATTACAGACAGGTGCCAACATCACCAG
CTGAATGTTGAGCGTTTAAGCAGATATTAAGTAAAGACACTGGCAGAGGG
TAGGAGTCCTGGGGATACTGAAGCACCTAGAGATGTCTTGGGCCTCTAGG
AGTGGGGTGAAGAGAGGAAACTGAAGCATGGAGGAAGGGCGTGGTATCTG
AGGATGTAGATGTGTAAGCCTGGCTAAGGAGCAGGGTGCAGGCCCCTCTC
TCAAACTAATGCAGATGCCTCCTATTAGCCAAACACACTGGAGGCTGGGA
GGCTGGTTGCTGTGGTCTGCAGGGCCAGTGCAAAGGCCAGGGATGGGAGC
AGAGGCCCCATGGCCAGCACTGGTATCCTGACTGGAGATTGACGGTACTA
AGATTCCTGACCACATCCCTGAAGCTAGGCATAACCTGACTCTCAGGGGA
GATGTGGAGCTCAGAATCCAGAGAGTGGAATAGAGAACCCTCCGAGCAGG
CATATAGATTCAGGGGCTGGAGTTACGGAACACCGTGCTCCCCAGCCAGA
GAGAAATGAGGACACTGGCCCCTGGTCTGTCTTCTGGGCCCCAGGAGGAA
GACTTTGTGAAGGCTGGGGAGGTGGACAGTCAGGTGGGGCTGCTGTGGGC
TGCTATTAGCTGAAGGGCTTTTGAAGCTAAGTGCATGGCTGTCTGGTTCT
GTAGGCCCTGAAGTTCCACAATGTAGGTTCCTGGCAGC

U6-cr437 (sequence encoding cr437 single guide RNA in bold underlined)

(SEQ ID NO: 16)

GAATTGATACTCGAGGGCCTATTTTCCCATGATTCCTTCATATTTGCATA
TACGATACAAGGCTGTTAGAGAGATAATTGGAATTAATTTGACTGTAAAC
ACAAAGATATTAGTACAAAATACGTGACGTAGAAAGTAATAATTTCTTGG
GTAGTTTGCAGTTTTAAAATTATGTTTTAAAATGGACTATCATATGCTTA
CCGTAACTTGAAAGTATTTCGATTTCTTGGCTTTATATATCTTGTGGAAA
GGACGAAACACCGCTGCCAGGAACCTACATTGGTTTTAGAGCTAGAAATA
GCAAGTTAAAATAAGGCTAGTCCGTTATCAACTTGAAAAAGTGGCACCGA
GTCGGTGCTTTTTTT

1. A vector system comprising one or more vectors encoding:
1) a nuclease system that cleaves a first target sequence on a target nucleic acid molecule, the nuclease system comprising at least one nuclease,
wherein the vector encoding the nuclease comprises a nucleotide sequence encoding the nuclease operably linked to a transcriptional or translational control sequence, and
2) a template sequence flanked at each end respectively by a second target sequence and a third target sequence that the nuclease system cleaves.

2. A vector system comprising one or more vectors encoding:
1) a nuclease system that cleaves a first target sequence on a target nucleic acid molecule, the nuclease system comprising at least one nuclease,
wherein the vector system encoding the nuclease comprises a nucleotide sequence capable of being translated into the nuclease, and
2) a template sequence flanked at each end respectively by a second target sequence and a third target sequence that the nuclease system cleaves.

3. The vector system of claim 2, wherein the vector system encoding the nuclease is an mRNA encoding the nuclease.

4. The vector system of claim 1, wherein the vector encoding the nuclease comprises two or more target sequences.

5. The vector system of claim 1, wherein the nuclease is a Cas nuclease.

6. (canceled)

7. The vector system of claim 1, wherein the nuclease is a Cas9 protein.

8. The vector system of claim 5, wherein the nuclease system further comprises at least one guide RNA that recognizes the first, second, or third target sequence.

9. The vector system of claim 8, comprising a first vector encoding the Cas9 protein, and a second vector comprising the template and a nucleotide sequence encoding the guide RNA operably linked to a second transcriptional or translational control sequence.

10. The vector system of claim 8, wherein the vector encoding the Cas9 protein further comprises the template and a nucleotide sequence encoding the guide RNA operably linked to a second transcriptional or translational control sequence.

11. (canceled)

12. The vector system of claim 5, wherein the first, second, and third target sequences are of the same nucleotide sequence, and wherein the nuclease system comprises a single guide RNA that recognizes the target sequences.

13.-29. (canceled)

30. A method for editing a target nucleic acid molecule in a eukaryotic cell, the method comprising administering the vector system of claim 1 to the cell.

31. (canceled)

32. The method of claim 30, wherein the cell is a human cell.

33. The method of claim 30, wherein the nuclease system cleaves the first target sequence on the target nucleic acid molecule in the eukaryotic cell, and the cleaved target nucleic acid molecule is repaired by homologous recombination with the template.

34. The method of claim 30, wherein the nuclease system cleaves the first target sequence on the target nucleic acid molecule in the eukaryotic cell, and the cleaved target nucleic acid molecule is repaired by homology-directed repair with the template.

35. The method of claim 30, wherein the nuclease system cleaves the first target sequence on the target nucleic acid molecule in the eukaryotic cell, and the template is inserted into the cleaved target nucleic acid molecule by non-homologous end joining.

36. A method for producing a virus comprising a nucleic acid, the method comprising:
providing a cell expressing a LacI protein,
introducing into the cell the nucleic acid,
introducing into the cell one or ore viral components for producing the virus, growing the cell, and
isolating the virus comprising the nucleic acid from the cell,
wherein the nucleic acid encodes:
1) a nuclease system that cleaves a first target sequence on a target nucleic acid molecule, the nuclease system comprising at least one nuclease,
wherein the nucleic acid comprises:
a nucleotide sequence encoding the nuclease operably linked to a first transcriptional or translational control sequence, and
at least two lacO sequences within the first transcriptional or translational control sequence or between the first transcriptional or translational control sequence and the nucleotide sequence encoding the nuclease, and
2) a template sequence flanked at each end respectively by a second target sequence and a third target sequence that the nuclease system cleaves.

37. (canceled)

38. The method of claim 36, wherein the LacI protein is fused with a KRAB domain.

39. The method of claim 36, further comprising adding an agent to remove the LacI bound to the lacO during or after isolation of the virus.

40. The method of claim 36, wherein the one or more viral components are encoded by the nucleic add.

41. The method of claim 36, wherein the one or more viral components are introduced via a separate vector other than the nucleic add.

42.-46. (canceled)
